(19) United States
(12) Patent Application Publication
(10) Pub. No.: US 2023/0234041 A1
(43) Pub. Date: Jul. 27, 2023

(54) SUPRAMOLECULAR POROUS ORGANIC NANOCOMPOSITES FOR HETEROGENEOUS PHOTOCATALYSIS

(71) Applicant: Northwestern University, Evanston, IL (US)

(72) Inventors: Yassine Beldjoudi, Wheeling, IL (US); Ahmet Atilgan, Chicago, IL (US); James Fraser Stoddart, Evanston, IL (US)

(21) Appl. No.: 17/996,744

(22) PCT Filed: Apr. 20, 2021
(86) PCT No.: PCT/US2021/070431

§ 371 Date: Oct. 20, 2022

Related U.S. Application Data
(60) Provisional application No. 63/012,642, filed on Apr. 20, 2020.

(51) Int. Cl. B01J 31/10; B01J 31/02; B01J 35/00; B01J 35/06; B01J 37/04; B01J 37/30; B01J 37/03; B01D 53/86; B01D 53/00

(52) U.S. Cl. CPC B01J 31/10; B01D 53/007; B01D 53/8687; B01J 31/0231; B01J 31/0284; B01J 35/004; B01J 35/06; B01J 37/04; B01J 37/30; B01J 37/031; B01D 2255/70; B01D 2255/802; B01D 2257/70; B01D 2259/4583

(57) ABSTRACT

Disclosed herein are supramolecular porous organic nanocomposites for heterogenous photocatalysis as well as methods of making and using the same. The nanocomposite comprises an admixture of a polymeric matrix and a macrocycle.

### CROSS-REFERENCE TO RELATED APPLICATIONS

This application claims benefit of priority to U.S. Pat. Application Ser. No. 63/012,642, filed Apr. 20, 2020, the contents of which are incorporated by reference in its entirety.

### FIELD OF THE INVENTION

The disclosed technology is directed to the utilization of supramolecular complexes to generate exciplexes that can be are utilized as efficient photosensitizers. More particularly the technology is directed to supramolecular porous organic nanocomposites for heterogeneous photocatalysis.

### BACKGROUND OF THE INVENTION

Considerable interest has been devoted towards the photo-oxidation of the sulfur mustard (SM) and 2-chloroethyl ethyl sulfide (CEES) using ¹O₂. The latter is a mild oxidant and photocatalysis has been proven to involve faster kinetics and also to be more selective when the less harmful sulfoxide derivative, 2-chloroethyl ethyl sulfoxide (CEESO), is formed as a major product with the 2-chloroethyl ethyl sulfone derivative (CEESO₂), as a minor product. Several porous materials, such as metal-organic frameworks (MOFs) and covalent organic frameworks (COFs), with photosynthesizing properties have been utilized for heterogenous photocatalysis of SM or CEES since the large surface areas of these porous materials facilitate the accessibility of the reactants to the photoactive sites. Processability of these crystalline powder materials towards military protective equipment (MPE), however, remains challenging. Recently, Karwacki et al.¹² reported efficient photocatalysis of CEES to CEESO using 4,4-difluoro-4-bora-3a,4a-diaza-s-indacene (BODIPY) photosensitizers doped into organic polymeric matrices. Nevertheless, the weak interactions through dispersive forces of the BODIPY photosensitizers to the polymer matrices hamper development of viable MPEs because of the leaching of the photosensitizer under catalytic conditions. In addition, a large amount of photocatalyst is required to decrease the conversion lifetime to < 1 min. As a result, there is a need for photocatalysts capable of photooxidizing SM and other harmful compound.

### BRIEF SUMMARY OF THE INVENTION

The present technology is directed to inexpensive, photostable, easily processed, and environment friendly porous organic polymeric nanocomposites and photocatalysts for removing contaminants or reactive substrates, such as chemical warfare agents, from the environment. The nanocomposites comprise an admixture of a polymeric matrix and a macrocyle. The composites are microporous and efficiently absorb electron rich molecules such as polyaromatic hydrocarbons. In some embodiments, the macrocycle is a cationic cyclophane, such as tetracationic ExBox⁴⁺ or Ex^{2.2}Box⁴⁺. In some embodiments, the polymeric matrix is an anionic polymer, such as polystyrene sulfonate (PSS).

The present technology is further directed to the generation of host/guest complex between the macrocycle and a polyaromatic guest which is dispersed within the polymeric matrix to form a photocatalyst. In some embodiments, the polyaromatic guest comprises 1,3,5,8-tetrabropyrene (TBP).

Under photoexcitation, the host-guest complexes shown efficient singlet oxygen (¹O₂) generation and therefore is an effective photocatalyst for a selective removal of contaminants from the environment. The efficient photocatalysis is associated with efficient intersystem crossing and the existence of a manifold of newly excited states that lead to the population of a locally excited state on the polyaromatic guest. Efficient intersystem crossing occurs because of a combination of spin-orbit coupling and a charge transfer between the guest and the host molecules.

These nanocomposites and photocatalysts are insoluble in organic and aqueous solvents which make them desirable for device fabrication. Accordingly, fibers, fabrics, or nanoparticles may be formed from any of the nanocomposites or photocatalysts described herein.

Another aspect of the technology is a method for photocatalytic oxidation of a reactive substrate. The method may comprise contacting any of the photocatalysts or the nanocomposites described herein with a reactive substrate and irradiating the photocatalyst or the nanocomposite in the presence of the reactive substrate, thereby oxidizing the reactive substrate. Suitably, the reactive substrate is a thioether or an organophosphorous compound, including without limitation chemical warfare agents.

Another aspect of the technology is a method for the generation of singlet oxygen (¹O₂). The method may comprise irradiating any of the photocatalysts or the nanocomposites described herein in the presence of an oxygen source. Suitably, the oxygen source is triplet oxygen (³O₂).

Another aspect of the technology is a method for sequestering an environmental contaminant. The method may comprise contacting any of the photocatalysts or the nanocomposites described herein with the environmental contaminant under conditions suitable to the adsorption of the environmental contaminant. Suitably, the environmental contaminate is a polyaromatic compound.

Another aspect of the technology is methods for preparing nanocomposites or photocatalysts. The method may comprise providing a first macrocycle solution comprising a macrocycle, a macrocycle solvent, and a first counterion, preparing a second macrocycle solution comprising the macrocycle, the solvent, and a second counterion, wherein the second counterion is different than the first counterion, providing a polymer solution comprising a polymer and a polymer solvent, mixing the second macrocycle solution and the polymer solution, thereby precipitating the nanocomposite or the photocatalyst from solution. In some embodiments, the first macrocycle solution and/or the second macrocycle solution comprises a host-guest complex comprising the macrocycle and a polyaromatic guest. In some embodiments, the second macrocycle solution is prepared by ion exchange between the first counterion and the second counterion.

These and other aspects of the technology will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows the photoinduced electron-exchange in a D-A dyad, acting as an efficient triplet photosensitizer.

FIG. 2A shows CO₂ sorption isotherms at 195 K for Na•PSS, ExBox•PSS and Ex^{2.2}Box•PSS

FIG. 2B provides cross-sectional SEM image showing the rough texture of the ExBox•PSS composite.

FIG. 2C provides cross-sectional SEM image showing the rough texture of the ExBox•PSS composite.

FIG. 2D provides cross-sectional SEM image showing the rough texture the TBP⊂ExBox•PSS composite.

FIG. 2E provides cross-sectional SEM image showing the rough texture the TBP⊂ExBox•PSS composite.

FIG. 3A shows diffuse reflectance spectra recorded for the TBP⊂ExBox•4PF₆ complex in 2-methyltetrahydrofuran (blue) and for the TBP (black), ExBox⁴⁺•PSS (green) and TBP⊂ExBox⁴⁺•PSS composite (red) in the solid state.

FIG. 3B shows excitation and emission spectra of the TBP⊂ExBox•PF₆ complex in MeTHF, recorded at 77 and 298 K.

FIG. 3C shows kinetic analysis of the femtosecond transient absorption data of TBP⊂ExBox•PF₆ at λₑₓ = 414 nm showing: i) fits to the solution of a 4-state kinetic model (A → B →C →D); ii) model populations as a function of time; iii) evolution-associated spectra for each species in the model.

FIG. 3D provides excitation and emission spectra of the TBP⊂ExBox•PSS composite.

FIG. 3E shows emission decay of the TBP⊂ExBox•PSS composite at λₑₓ = 405 nm fitted using a triple exponential fit.

FIG. 4 illustrates frontier orbitals of the TBP⊂ExBox⁴⁺ complex calculated from the APFD/6-31G(d) geometry optimized molecular structure. H and L represent respectively HOMOs and LUMOs.

FIG. 5A shows energy diagrams and transition configurations of singlet (Sₙ) and triplet (Tₙ) excited states of the TBP⊂ExBox⁴⁺ complex. Feasible excited states for exciton transformation are highlighted in colors. H and L represent respectively HOMOs and LUMOs.

FIG. 5B shows a schematic representation of the energy levels of the TBP⊂ExBox⁴⁺ complex highlighting the photosensitized singlet oxygen production.

FIG. 5C illustrates calculated HONTO and LUNTO distributions with population percentages are shown in red and blue colors for the singlet and triplet transitions respectively. The extents of the orbital overlap in the singlet (Sₙ) and triplet (Tₙ) states for exciton transformation in TBP⊂ExBox⁴⁺ are given in purple color.

FIG. 6A provides the structural formulas of the possible products of the photocatalysis of CEES with ¹O₂.

FIG. 6B shows homogeneous photocatalysis of CEES in MeOH using ExBox⁴⁺•4Cl, Ex^{2.2}Box•4Cl and the TBP⊂ExBox•4Cl complex.

FIG. 6C shows heterogeneous catalysis of CEES with 1% mole ExBox⁴⁺•PSS, Ex^{2.2}Box•PSS and the TBP⊂ExBox•PSS composites.

FIG. 7 provides diffraction X-ray pattern profiles of ExBox•PSS at room temperature.

FIG. 8 shows diffraction X-ray pattern profiles of Ex^{2.2}Box•PSS at room temperature.

FIG. 9 shows CO₂ Adsorption isotherm of ExBox•PSS at 278 K.

FIG. 10 provides pore sizes distribution in the ExBox•PSS composite calculated from the adsorption isotherm at 195 K.

FIG. 11 shows SEM image of a thin film of Na•PSS.

FIG. 12 provides UV-vis spectra of the TTF solution in MeOH before (black) and after (green) addition of the ExBox•PSS composite. After 60 min soaking of the composite in TTF solution, the concentration of TTF decrease significantly as a result of its diffusion inside the polymer composite.

FIG. 13 shows homogenous photocatalysis of CEES in CD₃OD using Ex^{2.2}Box⁴⁺•4Cl at 395 nm. The ratios of the different products have been estimated from the ¹H NMR integration peaks. Reference standard have been utilized.

FIG. 14 shows comparison of photocatalytic performance of structural components: Na•PSS, TBP and TBP-Na•PSS.

FIG. 15 shows heterogeneous photocatalysis using the TBP⊂ExBox•PSS: Comparison between the photoexcitation at 395 and 450 nm.

FIG. 16 shows photocatalytic stability of the TBP⊂ExBox•PSS composite was tested by leaching test at 50% (5 min) conversion of the CEES.

### DETAILED DESCRIPTION OF THE INVENTION

The present technology is directed to supramolecular porous organic nanocomposites for heterogenous photocatalysis as well as methods of making and using the same. The photocatalysts are inexpensive, photostable, easy to process, and environmentally friendly porous organic polymeric nanocomposites for removing contaminants from the environment, including photocatalysis of chemical warfare agents. The nanocomposites described herein comprise an admixture of a polymeric matrix and a macrocycle. The nanocomposites may form host-guest complexes between the macrocycle host and a guest that is dispersed within the polymeric matrix. These composites are insoluble in organic and aqueous solvents which make them desirable for device fabrication. In addition, these composites are microporous, therefore can adsorb polyaromatic pollutants. Under photoexcitation, host-guest supramolecular photocatalysts have shown efficient singlet oxygen (¹O₂) generation and therefore effective photocatalyst for a selective removal of contaminants from the environment. The photocatalysts show high photostability and reusability. These novel photocatalytic materials can be utilized as gels, powders, membranes, coatings, paints, filters, fibers, fabrics or textiles, personal protective equipment (such as masks), or materials for photodynamic therapy.

The present technology possesses a number of advantages that improves or differentiates it from photocatalysts known in the art. The nanocomposites are microporous and the macrocycles possess a permanent cavity which is efficient for hosting electron-rich compounds such as polyaromatic hydrocarbons. The composites may be transparent; therefore, more material can be utilized for photocatalysis. The nanocomposites and photocatalysts described herein are highly stable materials under photocatalytic process, biocompatible, environmentally friendly, easily processed for the fabrication of gels, porous membranes, coatings, and fibers. These amorphous polymeric nanocomposites materials are easy to process and can be included with textile polymers to develop protective clothes and equipment against chemical warfare agents or develop water purification filters and antimicrobial materials.

One aspect of the invention is a novel method for the preparation of the nanocomposites and photocatalysts described herein via counterion exchange between a macrocycle or porous cyclophane and polymer matrices. The strategies utilized so far for the preparation of porous materials are the covalent organic frameworks (COFs), metal-organic frameworks (MOFs), porous organic polymers (POPs) and polymer of intrinsic microporosity (PIMs). COFs and the MOFs often are difficult to prepare in large scale and are very complicated to process for device fabrication. In addition, COFs and MOFs are crystalline and it is difficult to control their structural integrity when incorporated within devices and other polymeric materials. Other COFs and MOFs structures collapse upon removal of solvent. POPs, requires extensive organic synthesis and required expensive rare-earth metal catalysts. All these materials are difficult to process for the development of large-scale equipment for water purification, or filtrations, and protective equipment against chemical warfare agents.

In contrast, the nanocomposites described herein are easily tunable to be (or not) soluble in water by changing the ratios of polymer to macrocycle. In addition, all these composites are amorphous which bypass the crystallinity problems often encountered in COFs and MOFs when applied into materials and devices. In addition, the composites can be incorporated into other polymers and fibers for the development of several porous materials.

In this context, development of sustainable photosensitizing organic materials for the heterogenous catalysis requires that the material fulfill these four main requirements — (i) it is porous and increases the photoactive surface area and facilitates the diffusion of reactant and products, (ii) ¹O₂ generation is efficient, (iii) the material is stable under photocatalytic conditions, and (iv) its preparation needs to be easy, inexpensive, scalable, and capable of being incorporated into products, such as MPE.

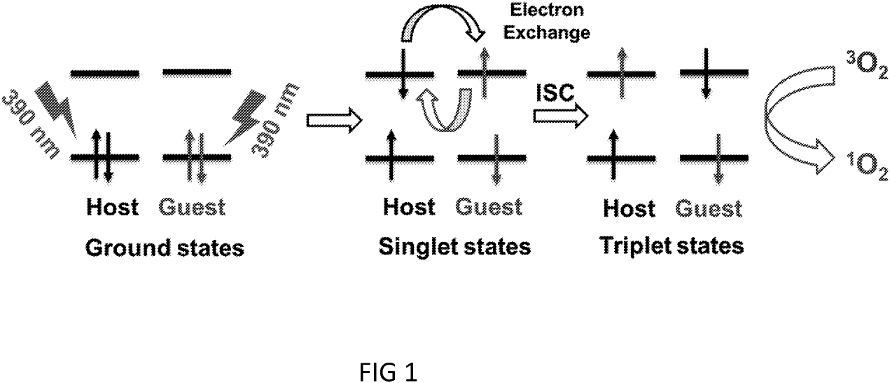

Another aspect of the disclosed technology is the use of host-guest supramolecular donor-acceptor dyads to enhance the photosensitizing performance. A polyaromatic guest may be utilized as an electron donor with macrocycle as the electron acceptor in order to form a host-guest D-A supramolecular complex. This complex promotes the S-T exciton transformation between the two excited states of the two components (FIG. 1), enhancing ISC to populate the low-lying locally excited (LE) triplet state (T₁) of the guest or macrocycle. This design strategy requires (i) efficient CT between the guest (D) and host (A) (ii) the two fluorophores absorb similar radiation wavelengths in order to access the excited-states of both chromophores, (iii) a small ΔE_{ST} (< 0.37 eV) and small distance between the D and A in order to facilitate spin-orbit charge-transfer intersystem crossing¹⁹ (SOCT-ISC), (iv) the energies of both CT and T₁ states must be similar, and (v) incorporation of heteroatoms in the host and/or the guest can facilitate the S-T transformation and offer a low lying triplet state that can promote energy transfer to molecular oxygen.²⁰

Although compounds, such as TBP which is used in the Examples, may have a low-lying triplet state (T₁, 1.89 eV) close in energy to the molecular oxygen (1.63 eV) facilitating the energy transfer to generate the singlet oxygen, the inefficient intersystem crossing and internal conversion may hamper the population of the T₁ triplet state. Host-guest complexes may have a manifold of excited states involving, locally excited states, charge transfer states, and hybrid locally charge transfer states that enhance not only the intersystem crossing mechanism but also the decay from the upper states through internal conversion mechanisms. As demonstrated in the Examples, the photosensitizers prepared according to the presently disclosed technology efficiently generate singlet oxygen. Previous studies, in contrast, have been limited into the development of intramolecular donor-acceptor dyads as efficient photosensitizers. Another aspect of the invention is the incorporation of the host-guest photosensitizer into polymer matrices for the preparation of heterogenous photocatalysts. Although, non-porous polymers have been used to prepare singlet oxygen photosensitizer thin films, leaching of photosensitizer, the lack of porosity in the materials prepared in this fashion, and aggregation of photosensitizers inhibit development of these materials for use protective equipment against chemical agents, such as sulfur mustard.

The nanocomposites and photocatalysts described herein comprise an admixture of a polymeric matrix and a macrocycle. “Macrocycle” refers to a cyclic macromolecular or a macromolecular cyclic portion of a macromolecule. “Macromolecule” refers to a molecule of high relative molecular mass, the structure of which essentially comprises the multiple repetition of units derived, actually or conceptually, from molecules of low relative molecular mass. In some embodiments, the macrocycle is cyclophane. The incorporation of the macrocycle into the polymeric matrix allows for the material to have an intrinsic porosity.

“Cyclophane” refers to compounds having (i) mancude-ring systems, or assemblies of mancude-ring systems, and (ii) atoms and/or saturated or unsaturated chains as alternate components of a large ring. “Mancude-ring systems” refers to rings having (formally) the maximum number of noncumulative double bonds, e.g. benzene, indene, indole, 4H-1,3-dioxine. Exemplary cyclophanes include ExBox⁴⁺ or Ex^{2.2}Box⁴⁺.

“Polymeric matrix” refers to a polymer capable of surrounding the macrocycle and interacting with the macrocycle to prepare nanocomposites. In some embodiments, the polymeric matrix may non-covalently interact (for example via electrostatic, van der Waals, π-π interaction, or the like) with the macrocycle to prepare stable nanocomposites where neither the polymeric matrix nor macrocycle substantially leeches into solution when immersed into a solvent. In some embodiments, the polymeric matrix may be transparent or substantially transparent in a desired spectral window.

Exemplary polymeric matrixes include anionic polymers, biopolymers or natural polymers, polymers suitable for 3D printing of plastics. For example, anionic polymers may include sulfate polysaccharides (heparin, mannan sulfate, dextran sulfate and chondroitin sulfate) and starch with carboxylic substitutions. Biopolymers or natural polymers may include, for example, proteins, polynucleic acids, poly lactic acid, polyglyconic acid, poly-3- hydroxybutyrate, cellulose, chitosan, guar gum, starch, tannin and sodium alginate for the development of composites for biomedical applications. Polymers suitable for 3D printing of plastics may include polylactic acid or polyethylene terephthalate. Other exemplary polymers for use with the present technology include, without limitation, polystyrene sulfonate (PSS), cellulose acetate (CA), polyamide (PA), polyvinylidene fluoride (PVDF), polysulfone (PSF), polyethersulfone (PES), polyvinyl chloride (PVC), polyimide (Pl), polyacrylonitrile (PAN), polyethylene glycol (PEG), polyvinyl alcohol (PVA), poly(methacrylic acid) (PMAA), poly(arylene ether ketone) (PAEK), poly(ether imide) (PEI), polyaniline nanoparticles (PANI), sulfonated poly(arylene ether sulfone) (SPAES), and the like.

The macrocycles of the present invention may be charged to allow for electrostatic interactions with the polymeric matrix. Suitably the macrocycles are cationic such as those that may be prepared from pyridinium subunits but other cationic or anionic subunits may also be employed to prepare the macrocycle.

In some embodiments, the nanocomposite comprises a photosensitizer. “Photosensitization” referrers to a process by which a photochemical or photophysical alteration occurs in one molecular entity as a result of initial absorption of radiation by another molecular entity called a photosensitizer. Suitably, the photosensitizer is not consumed in the reaction.

The photosensitizer may comprise a host-guest complex comprising the macrocycle and a guest. The guest suitably absorbs the same or substantially similar wavelength as the host macrocycle and has efficient intersystem crossing between the guest and host. In some embodiments, the guest is insoluble to avoid leaching of the guest from the macrocycle and the nanocomposite.

Suitably the guest is a polyaromatic guest. Polyaromatic guests comprise two or more fused aromatic rings. In some embodiments, the polyaromatic guest comprises one or more heavy atoms. As used herein, a heavy atom may include any atom heavier than carbon or, in some embodiments, heavier than fluorine or chlorine. Exemplarily polyaromatic guests include, without limitation, tetrabromopyrene (TBP), naphathalene diimide, or perylene diimide. Utilization of two organic dyads of similar excited state energies (similar exciton energies) leads to the increase of the efficiency of the intersystem crossing by combining both the spin-orbit charge transfer-intersystem crossing (SOCT-ISC) and spin orbit coupling (SOC) associated with the heavy atoms between the excited states. Therefore, the host-guest complex is a more efficient photo-synthesizer comparing to the performance of the individual component. This strategy offers great advantages since it does not require significant organic synthesis to prepare a donor-acceptor dyad. The present technology is versatile and macrocycles absorbing visible light or near-lR light can be used in combination with other guest molecules absorbing similar wavelengths to form a supramolecular Donor-Acceptor dyad.

The present technology may be used in a number of different applications. In one embodiment the nanocomposites and photocatalysts disclosed herein may be used to prepare gels, powders, membranes, coatings, paints, filters, fibers, fabrics or textiles, personal protective equipment (such as masks), or materials for photodynamic therapy. Suitably, the nanocomposites and photocatalysts can be incorporated into woven and nonwoven fibrous materials, such as wool felt, fiberglass paper, polypropylene, and so forth, or into polymers, such as polyester, polyamide, wool, and so forth. These materials can be used to develop textiles, clothes, masks, filters with photosensitizing properties for sequestering environmental contaminants, catalytically degrading contaminants, or killing or inhibiting the proliferation of microbes.

In another embodiment, the nanocomposites and photocatalysts disclosed herein may be used for photocatalytic oxidation of reactive substrates. The method may comprise contacting any of the nanocomposites and photocatalysts disclosed herein with a reactive substrate and irradiating the nanocomposite or photocatalyst in the presence of the reactive substrate, thereby oxidizing the reactive substrate. Suitably the reactive substrate may be a thioether or organophosphorous compound, which may be generally recognized as being a chemical warfare agents such as a sulfur mustard. Exemplary chemical warfare agents include, without limitation, VX, Soman, Sarin, Tabun, cyclosarin, mustard, and the like.

In another embodiments, the nanocomposites and photocatalysts disclosed herein may be used for the generation of singlet oxygen (¹O₂). The method may comprise irradiating any of the nanocomposites and photocatalysts disclosed herein in the presence of an oxygen source. Suitably, the oxygen source is triplet oxygen (³O₂) but other sources of oxygen may also be used. Suitably, the singlet oxygen may be used for photodynamic therapy. In another use, the singlet oxygen may be used to kill or inhibit the proliferation of microbes, such as viruses and bacteria, suitable for the purification of water or other liquids.

In another embodiments, the nanocomposites and photocatalysts disclosed herein may be used for sequestering an environmental contaminant. The method may comprise contacting any of the nanocomposites and photocatalysts disclosed herein with the environmental contaminant under conditions suitable to the absorption of the environmental contaminant. Suitably, the environmental contaminant is a polyaromatic compound, but other compounds may also be sequestered. Suitably, the environmental contaminant may be reversibly released by changing, for example, the redox state of the macrocycle. This allows for the preparation of recyclable materials for trapping or filtering environmental contaminants, such as polyaromatic compounds.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Molecular oxygen may be applied in the detoxification of Chemical Warfare’s Agents (CWAs), such as Sulfur Mustard (SM). Efficient heterogenous photosensitizing materials need to present both large accessible surface areas and excitons of suitable energies and with well-defined spin structures. Confinement of the tetracationic cyclophane (ExBox⁴⁺) within a non-porous anionic polystyrene sulfonate (PSS) matrix leads to a surface area increase of up to 225 m².g⁻¹ in ExBox•PSS. Our approach to enhancing the intersystem crossing (ISC) involves combining (i) efficient spin-orbit coupling (SOC) associated to lone-pair electrons of heavy atoms (Br atoms) in the 1,3,5,8-tetrabropyrene (TBP), and (ii) photoinduced electron transfer in a TBP⊂Exbox⁴⁺ supramolecular donor-acceptor (D-A) dyad to trigger a spin-orbit charge transfer ISC. The TBP⊂Exbox⁴⁺ complex displays a charge transfer band at 450 nm and an exciplex emission at 520 nm (λₑₓ = 380 nm, (Φ_{F} < 3%) with a short life time (< 1 ns) in both solution and in the solid state, indicating the formation of new mixed-electronic states between the D and A. Time-dependent DFT calculations have revealed that the efficient singlet-triplet (S-T) transformation is the result of the formation of a hybrid locally charge transfer (HLCT) excited state in the D-A complex and the close energy levels with the same transition configurations. The lowest triplet state (T₁, 1.89 eV) is a locally excited (LE) state on the TBP and close in energy with the charge separated state (CT, 2.14 eV). Transient absorption spectroscopy exciting the HLCT state at 414 nm shows the population of an emissive CT state followed by recombination to a long-lived triplet state (> 1.5 µs). The photocatalytic activities of the TBP⊂Exbox•4Cl and TBP⊂Exbox•PSS in homogenous and heterogenous media respectively for the conversion of a sulfur mustard simulant to its non-toxic sulfoxide analogue, has proved to be significantly more efficient than TBP and

ExBox⁴⁺, confirming the importance of the newly formed excited-state manifold in TBP⊂Exbox⁴⁺ for the population of low-lying T₁state. The high stability, inexpensiveness, facile preparation, and high performance of the TBP⊂ExBox•PSS complex augur well the future development of new supramolecular heterogenous photosensitizers using host-guest chemistry.

Here we describe (Scheme 1) the preparation of supramolecular porous organic composites using anionic polymeric matrices such as Polystyrene Sodium Sulfonate (Na•PSS) and extended tetracationic cyclophanes such as ExBox⁴⁺ and Ex^{2.2}Box⁴⁺. The rigidly defined cavities of these cyclophanes, when assembled within a polymeric matrix relying electrostatic interactions offers porous properties that are necessary in order to optimize the diffusion of reactants and products within them and increase the active surface area for ¹O₂ generation. Furthermore, tetracationic cyclophanes such as ExBox⁴⁺ are attractive candidates for ultrafast intermolecular CT from an electron-rich guest,¹³ intramolecular through-bond CT from thep-xylylene bridges to the extended bipyridinium units¹⁴ and multielectron accumulation,¹⁵ leading to an array¹⁶ of accessible mixed-valence states, and energy transfer from ExBox⁴⁺. Other investigations reported¹⁷ that the close interaction and the significant orbital overlap between the PDI (perylene diimide) as a guest and Exbox⁴⁺ acting as a host, enables ultrafast energy transfer to proceed by the electron exchange Dexter mechanism.¹⁸ In addition, incorporation of heavy atoms into the cyclophane leads to an efficient quenching of the fluorescence as a result of efficient spin-orbit ISC pathways leading to the generation of the triplet state on the PDI guest.¹⁶

Scheme 1. (top) The structural formals of building blocks utilized in the design of supramolecular photosynthesizing porous organic polymer for the a heterogenous photocatalysis. (a) ExBox⁴⁺, (b) Ex^{2.2}Box⁴⁺, (c) 1,3,6,8-tetrabromopyrene (TBP) (d) Sodium Polystytene Sulfonate (Na•PSS). (e) Synthesis of the TBP⊂ExBox•4PF₆, TBP⊂ExBox•4Cl and TBP⊂ExBox•PSS composites.

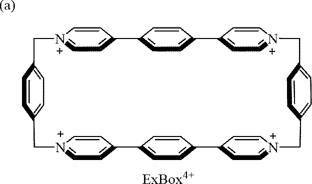

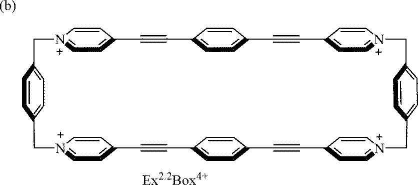

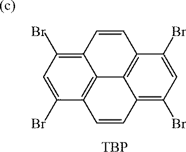

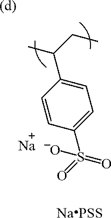

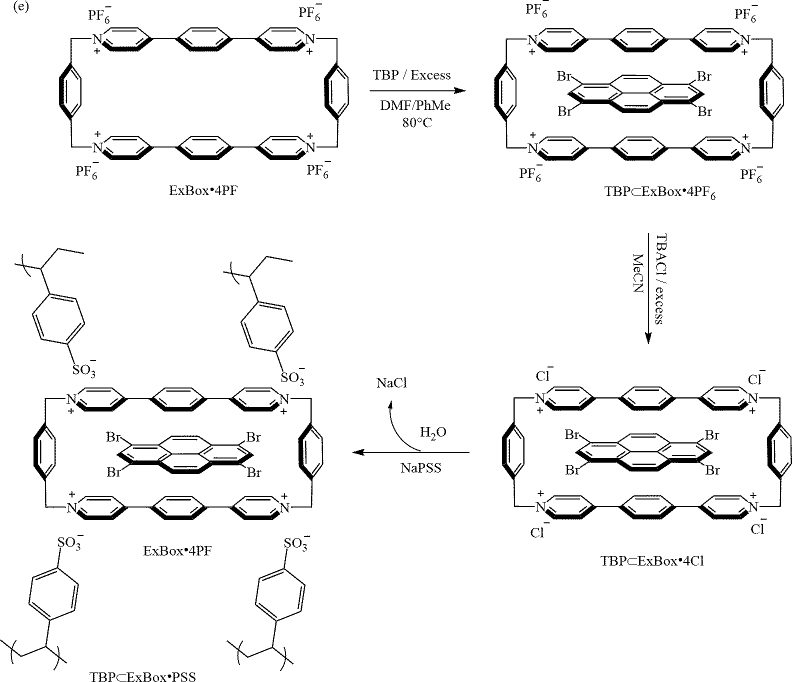

1,3,6,8-tetrabromopyrene (TBP) is utilized as an electron donor with ExBox⁴⁺ as the electron acceptor in order to form a host-guest D-A supramolecular complex (TBP⊂ExBox⁴⁺). This complex promotes the S-T exciton transformation between the two excited states of the two components (FIG. 1), enhancing ISC to populate the low-lying locally excited (LE) triplet state (T₁) of TBP. This design strategy requires (i) efficient CT between the guest (D) and host (A) (ii) the two fluorophores absorb similar radiation wavelengths in order to access the excited-states of both chromophores, (iii) a small ΔE_{ST} (< 0.37 eV) and small distance between the D and A in order to facilitate spin-orbit charge-transfer intersystem crossing¹⁹ (SOCT-ISC), (iv) the energies of both CT and T₁ states must be similar, and (v) incorporation of heteroatoms (N atoms) in the host (ExBox⁴⁺) and the guest (Br atoms) which can, not only facilitate the S-T transformation but also offer a low lying triplet state that can promote energy transfer to molecular oxygen.²⁰ (¹∑ = 1.63 eV).

From a practical perspective, the very low solubility of the TBP in organic and aqueous media at ambient temperatures is necessary in order to enhance the stability of the supramolecular photocatalyst since host-guest formation is not in equilibrium. It was previously reported²¹ that the water soluble cobalt(III) tetrahedral coordination capsules exhibit non-equilibrium guest binding properties because of the hydrophobic effect which is associated with the low solubility of the guest molecules in aqueous media. Finally, incorporation of the tetracationic TBP⊂ExBox⁴⁺ photosensitizer within the anionic matrix of PSS leads to the formation of a stable and porous composite for the heterogenous photocatalysis of CEES. All compounds have been characterized in solution and in the solid state by absorption, diffuse reflectance and fluorescence spectroscopies. Furthermore, the electronic properties of the host-guest complex have been unraveled using transient absorption spectroscopy and time-dependent DFT calculations. Finally, we have investigated the photocatalytic performance of TBP⊂ExBox⁴⁺•4PF₆, and TBP⊂ExBox⁴⁺•PSS for the elimination of the sulfur mustard simulant (CEES) in both homogenous and heterogenous media.

Preparation and Characterization. The ExBox•4PF₆ and Ex^{2.2}Box•4PF₆ cyclophanes were synthesized following the protocols already reported in literature²². Although TBP is insoluble in the most common organic solvents, at high temperatures it becomes soluble in PhMe to afford a pale yellow solution. The host-guest complex TBP⊂ExBox•4PF₆ can be formed (Scheme le) by dropwise addition of TBP, solubilized in hot PhMe into a solution of ExBox•4PF₆ in hot dimethyl formamide (DMF). After heating the mixture for 24 h at 80° C., an intense yellow/orange colored solution is formed. After evaporation of the solvent and solubilization of TBP⊂ExBox•4PF₆ in MeCN, the insoluble excess of TBP can be removed by filtration. Tetrabutylammonium chloride was added to the MeCN solution containing TBP⊂ExBox•4PF₆ in order to exchange the PF₆⁻ to Cl⁻ anions, a process that renders the cyclophanes soluble in aqueous media. After isolation of the TBP⊂ExBox•4Cl complex as a yellow powder, it was dissolved in H₂O and Na•PSS was added dropwise under strong agitation to form (Scheme le) a precipitate of TBP⊂ExBox•PSS composite of 5/3 w/w ratio. The very low solubility of the TBP, combined with the trapping of TBP⊂ExBox⁴⁺ within the PSS polymer matrix as a result of electrostatic interactions, is essential in order to enhance the stability of the composite in both aqueous and organic media with efficient heterogenous photocatalysis.

In order to ascertain the role of the host-guest D-A complex in the photocatalytic performances, the ExBox•PSS and Ex^{2.2}Box•PSS composites have also been prepared (Scheme 2 and 3) quantitatively following similar protocols. After ExBox•4Cl and Ex^{2.2}Box•4Cl have been dissolved in H₂O and Na•PSS was added dropwise to form the ExBox•PSS and Ex^{2.2}Box•PSS composites at 1/1 and 3/2 w/w ratios, respectively. These composites are insoluble in both aqueous and non-aqueous media. In order to study the optical properties of the composites in aqueous solutions, we prepared the ExBox•PSS and Ex^{2.2}Box•PSS composites at ⅓ and 1:1 w/w ratios, respectively.

Scheme 2: Preparation of ExBox•PSS composite

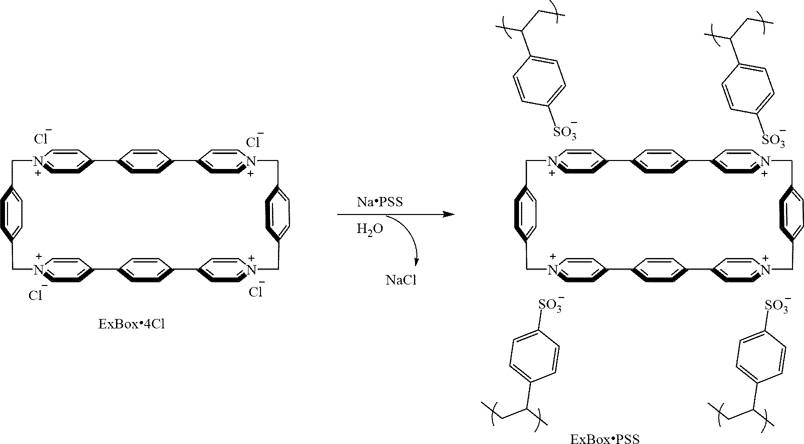

Scheme 3: Preparation of ExBox^{2.2}•PSS composite

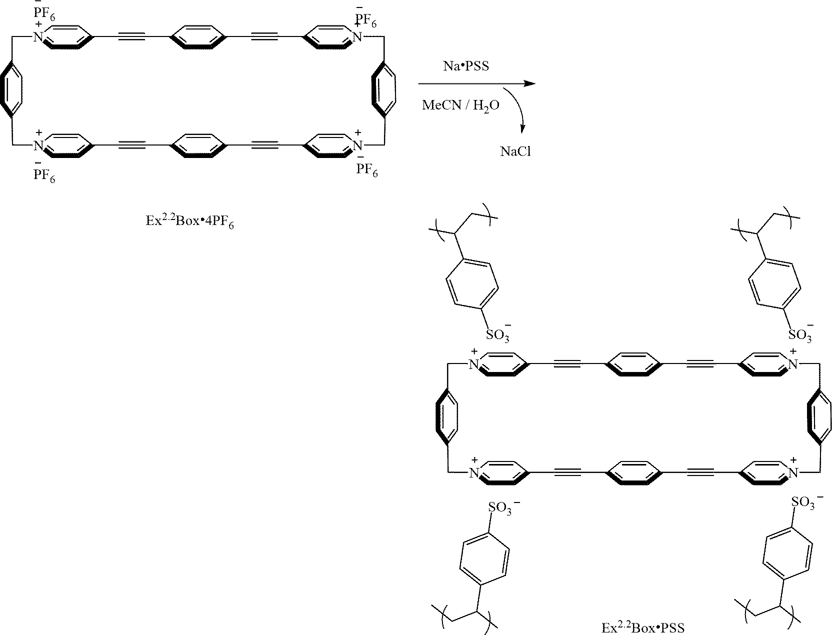

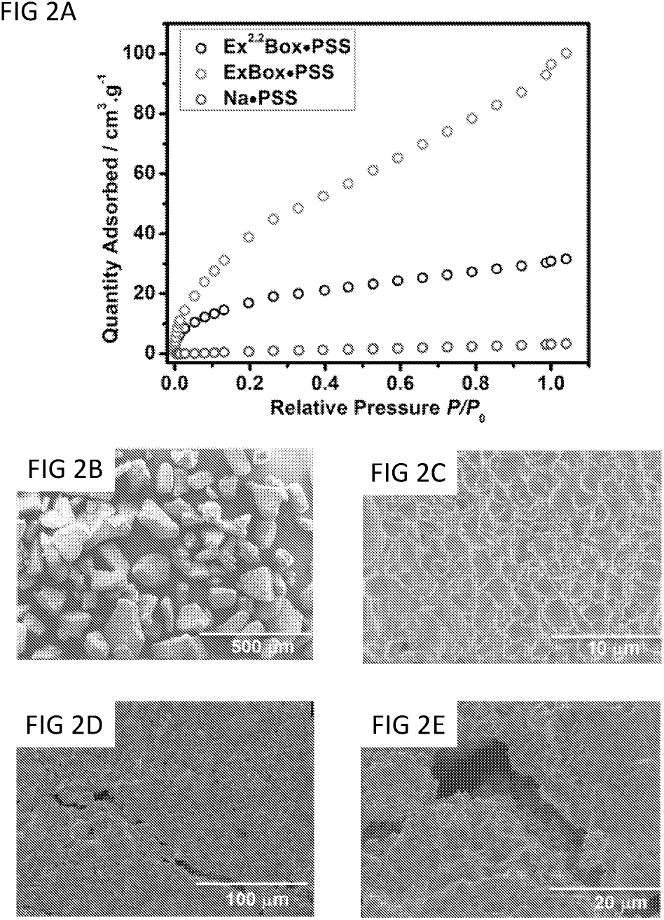

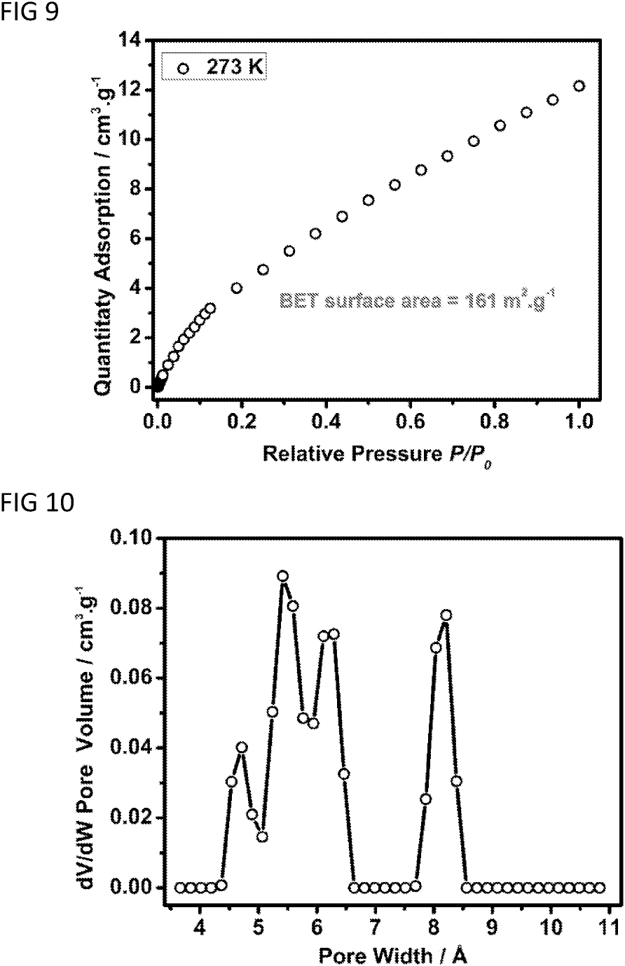

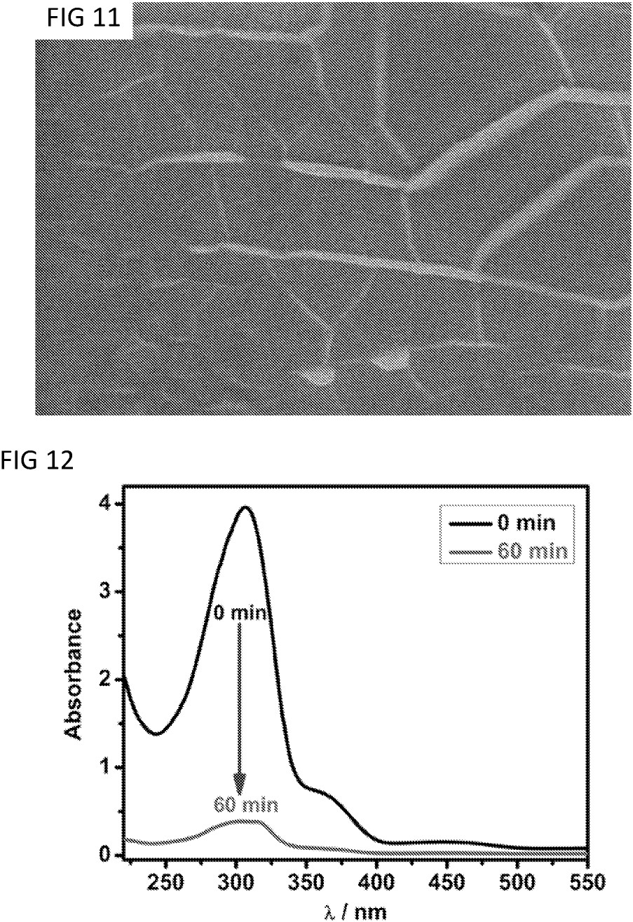

Sorption and Morphological Investigations. The CO₂ adsorption on the ExBox•PSS and Ex^{2.2}Box•PSS composites has been performed and compared to the adsorption isotherm of the pristine Na•PSS in order to confirm the role of the tetracationic cyclophanes in forming the porous nature of these composites. Furthermore, the surface area and the porosity of the ExBox•PSS were measured at 195 K and 295 K (FIGS. 2A and 11A) in order to confirm the persistence of the porosity of the composite at room temperature. FIG. 2A shows, as expected, a negligible adsorption of CO₂ into Na•PSS at 195 K indicative of its non-porous nature. The rapid increase in the CO₂ uptake at low pressures for both ExBox·PSS and Ex^{2.2}Box•PSS indicates the presence of micropores, whilst the continuous increase of the uptake confirms the presence of larger pores. The pore volume plot revealed (FIG. 10) the existence of several pores of different sizes, e.g., medium-sized micropores (7-9 Å) and ultra-micropores (<7 Å). These pore sizes are like those of other porous materials, such as MIL-47 and TIF-1 which possess²³ pore sizes in a range of 7-9 Å, while the MFI and MOR have pore sizes of <7 Å. Other polymers of intrinsic microporosity (PIMs) have been reported in the literature and exhibit similar pore sizes.²⁴ The Brunauer-Emmett-Teller (BET) surface areas of ExBox•PSS and Ex^{2.2}Box•PSS at 195 K were found (FIG. 2A) to be 226 m².g⁻¹ and 86 m².g⁻¹ respectively. Clearly, the inherent cavities in the tetracationic cyclophanes, combined with their distribution within an anionic polymeric matrix, leads to the increase in the surface area of the PSS matrix.

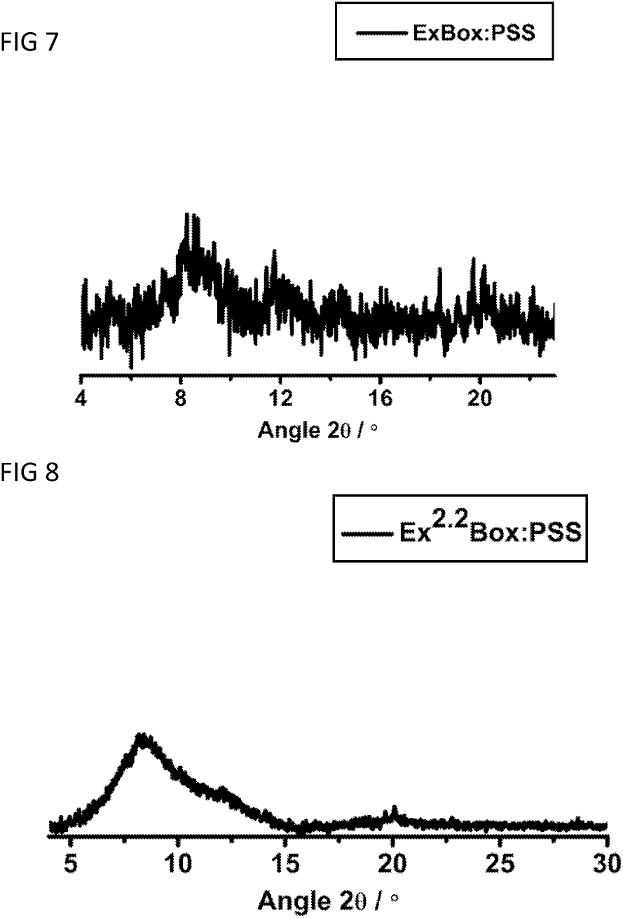

In order to test the diffusion of larger molecules, we investigated the adsorption of the tetrathiafulvalene (TTF) inside the ExBox·PSS composite. Previous studies have revealed²⁵ that TTF has a relatively strong affinity for the tetracationic cyclophanes, forming dark green host-guest complexes. Incorporation of the ExBox•PSS composite within a solution of the TTF of concentration of 10⁻⁵M led to the absorption of the TTF molecules, affording (FIG. 12) a dark green composite as a result of the CT interactions between the TTF and the ExBox⁴⁺ in the TTF⊂ExBox⁴⁺ host-guest complex. We conclude that the composite possesses large pores which allow the diffusion of both the reactant (CEES, O₂) and product (CEESO) molecules for photocatalytic applications. In recent years, considerable interest has been focussed²⁶ towards the use of porous materials for catalytic applications on account of their high active surface areas and low diffusion barriers. Scanning electron microscopy (SEM) has revealed that, while the Na•PSS (FIG. 11) has a smooth texture, the composites ExBox•PSS, Ex^{2.2}Box•PSS and TBP⊂ExBox•PSS are all characterized (FIGS. 2B-2E) by having a rough and spongy texture indicative of their porous natures. Powder XRD has revealed (FIGS. 7-8) that all the composites are amorphous, confirming the distribution of the tetracationic cyclophanes in the PSS matrix and the absence of phase separation between the Na•PSS and the cyclophanes.

### Photophysical Investigations

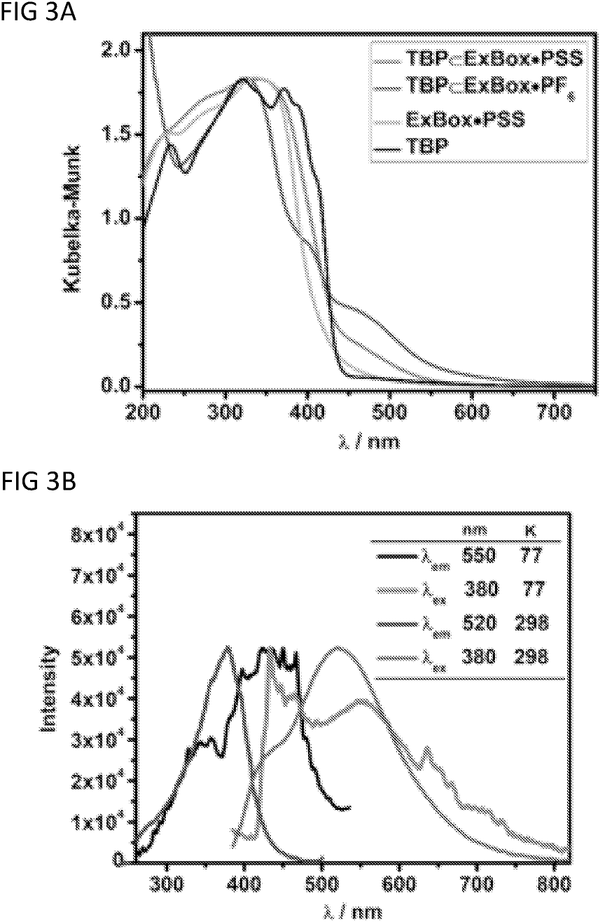

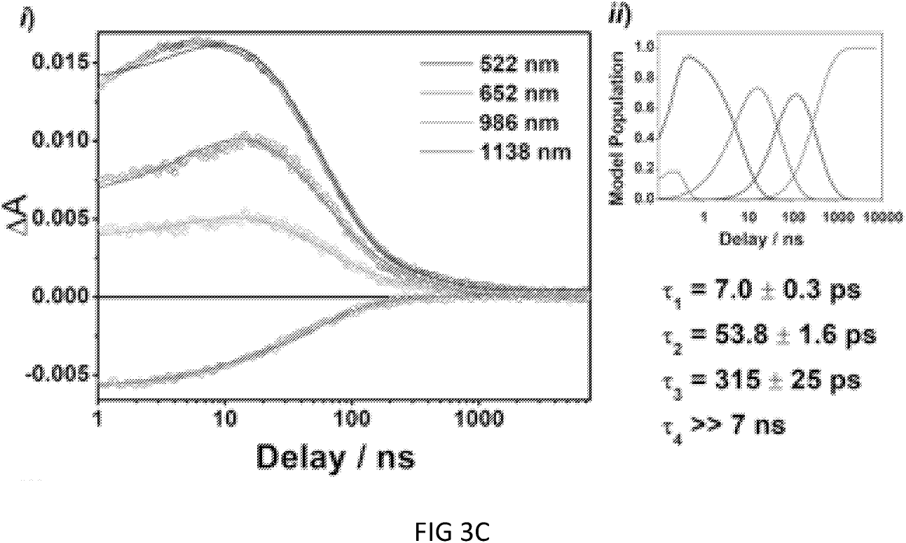

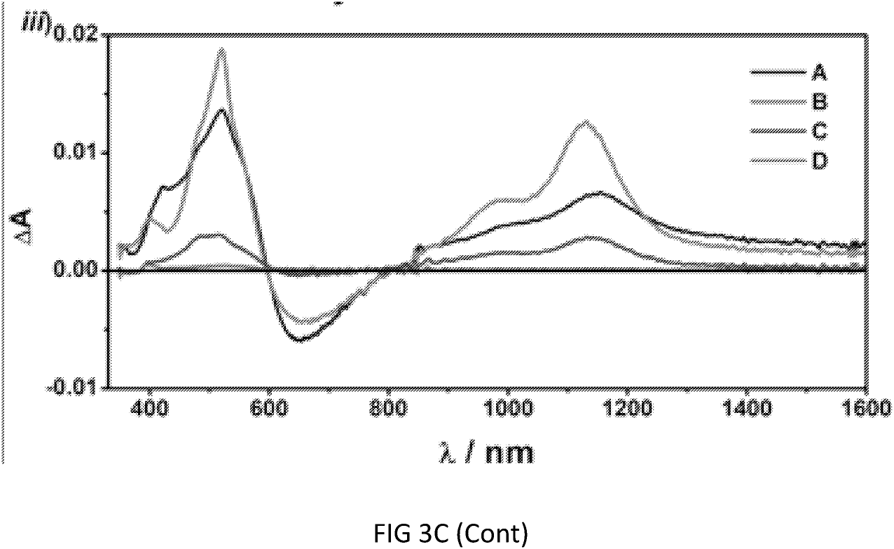

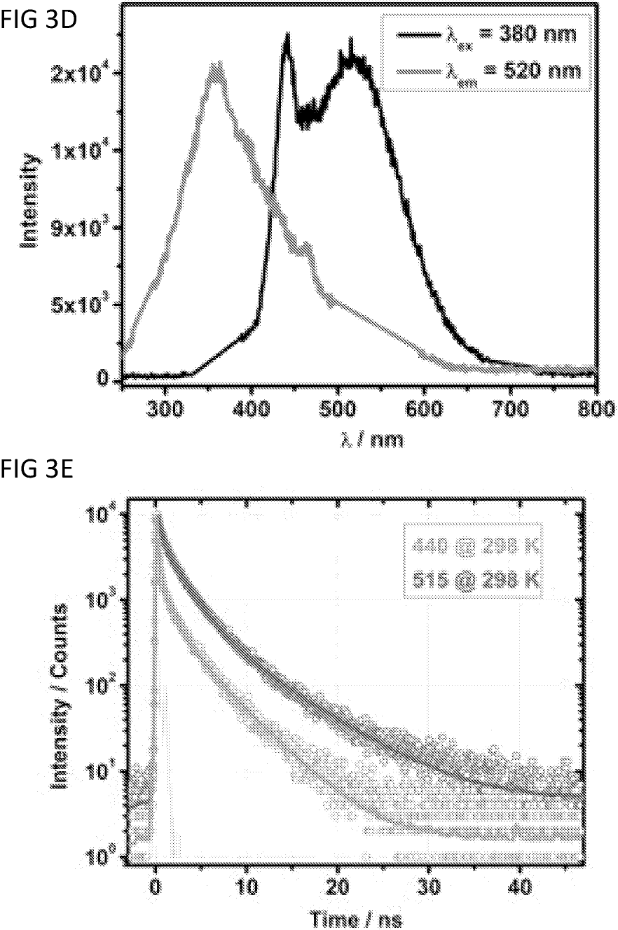

Solution Studies: Steady-State Spectroscopy: Absorption and fluorescence investigations have been carried out in order to unravel the electronic properties of the host-guest complex (TBP⊂ExBox•PF₆) in solution and the polymer composites in the solid-state. Na•PSS is colorless in H₂O and the UV-Vis absorption profile is characterized by the existence of two absorption bands at 223 and 252 nm, while fluorescence spectroscopy has shown that excitation at 254 nm offers a single emission band at 308 nm. ExBox•4Cl in H₂O displays excitation and emission bands at 358 and 383 nm, respectively, arising from the lowest singlet excited state. The Exbox•PSS composite of 1:3 w:w ratio is soluble in H₂O and displays the characteristic absorption features of ExBox⁴⁺ and PSS. The emission of this composite in aqueous solution exhibits a slight bathochromic shift of 47 nm to become centered at 430 nm as a consequence of the change in the polarity and viscosity of the media. Time-resolved photoluminescence decay was monitored at 430 nm, using 374 nm as the excitation wavelength. The decay curve was fitted to the double-exponential function, resulting in a slow component (τ₁ = 1.43 ns) and a fast one (τ₂ = 0.47 ns). In PhMe, TBP is weakly soluble and the absorption profile of TBP shows several absorption bands at 378, 359, 341 and 293 nm characteristic of the π→π_{*} and n→π_{*} transitions. The diffuse reflectance of TBP reveals (FIG. 3A) the existence of two maxima at 320 and 380 nm, similar to the solution absorption profile. Upon excitation at 380 nm, TBP offers a featured emission band in a range 400-470 nm (λₘₐₓ = 439 nm, (Φ_{F} = 1.62 %) with a Stokes shift (Table 6) of 0.22 eV. The singlet excited-state lifetime (Table 1) is rather long (τ₁= 0.11 ns, τ₂ = 0.60 ns, τ₃ = 10.27 ns), which is associated with the excimer emission as the result of the [π^{...}π] interactions. Whilst at 298 K the excitation band is centered on 377 nm associated with the existence [π^{...}π] interaction in the ground state, at 77 K, an intense excitation band appears at 315 nm with a smaller broad band in a range 340-420 nm. The incorporation of the TBP inside the cavity of the ExBox⁴⁺ does not affect significantly the ground-state electronic properties of either component. Indeed, the absorption spectra of TBP⊂ExBox•4Cl and TBP⊂ExBox•4PF₆ are characterized by the overlap of the absorption bands of both the host and the guest components with a maximum absorption centered on 320 and 358 nm in MeCN and H₂O, respectively. The diffuse reflectance spectrum of the TBP⊂ExBox•4PF₆ reveals (FIG. 3A) the existence of a CT broad band centered on 455 nm. It follows that the TBP⊂ExBox⁴⁺ supramolecular D-A dyads might exhibit a SOCT-ISC in order to enhance the exciton transformation. Previous investigations, have reported²⁷ that efficient ISC can be obtained with intramolecular electron D-A dyads, displaying n-π* ↔π-π* systems because the electron transfer (charge separation or recombination) results in will result in magnetic torque on the electron spin which will induce a molecular orbital angular momentum change, enhancing ISC. Steady-state fluorescence spectroscopy revealed (FIG. 3B, Table 1) the existence of two emission bands centered on 440 and 512 nm at 298 K. The band at 440 nm can be attributed to TBP monomer emission, while the one at 512 nm is considered to be an exciplex emission (¹S_{1CT}) arising from the TBP⊂ExBox⁴⁺ host-guest complex.

Transient Absorption Spectroscopy: The electronic properties of the TBP⊂ExBox•4PF₆ complex have also been investigated with transient absorption spectroscopy. Exciting at either 414 or 450 nm, the kinetics of the charge separation and recombination for TBP⊂ExBox⁴⁺ were obtained. See FIG. 3C. In addition, excitation at each of these wavelengths allows one us to be able to deconvolute the roles of the LE and CT states in the overall electronic properties. Indeed, on excitation at 414 nm the LE state TBP can be accessed, while at 450 nm only the lowest CT state can be reached. Photoexcitation of TBP⊂ExBox⁴⁺ at 414 or 450 nm results (FIG. 3C) in the appearance of strong peaks at 522, 985, and 1140 nm as well as, a radiative recombination band at 655 nm. Similar absorption bands have been observed¹² in Perylene⊂ExBox⁴⁺ CT complex without the radiative recombination band. This radiative recombination gives an estimate of the energy of the CT state of 1.89 eV. The DFT-calculated energy of the lowest triplet state (T₁) state is also 1.89 eV implying that these states may interact via SOCT-ISC. When excited at 414 nm, these bands are formed (FIG. 3C) immediately and rise over the next ~7ps, then decay in ~54 and ~300 ps. The immediate appearance of the bands associated with ExV^{+•} indicates that CT from the LE state of TBP is ultrafast (<300 fs), as it is the case of Perylene⊂ExBox⁴⁺. The ~7 ps time constant is associated with a structural relaxation of the charge-separated state,¹³ and the biexponential decay of the TBP⁺⊂ExBox³⁺ state is most likely a consequence of the distribution of binding geometries in solution. Both recombination processes are slightly longer than the ~40 ps charge recombination observed for Perylene⊂ExBox⁴⁺. Notably, direct excitation of the CT band (λₑₓ = 450 nm) offeres a similar TA profile, however, with generally longer time constants― a similar rise with ~8.6 ps, then decay in ~71 ps and a minor component decay in ~900 ps.

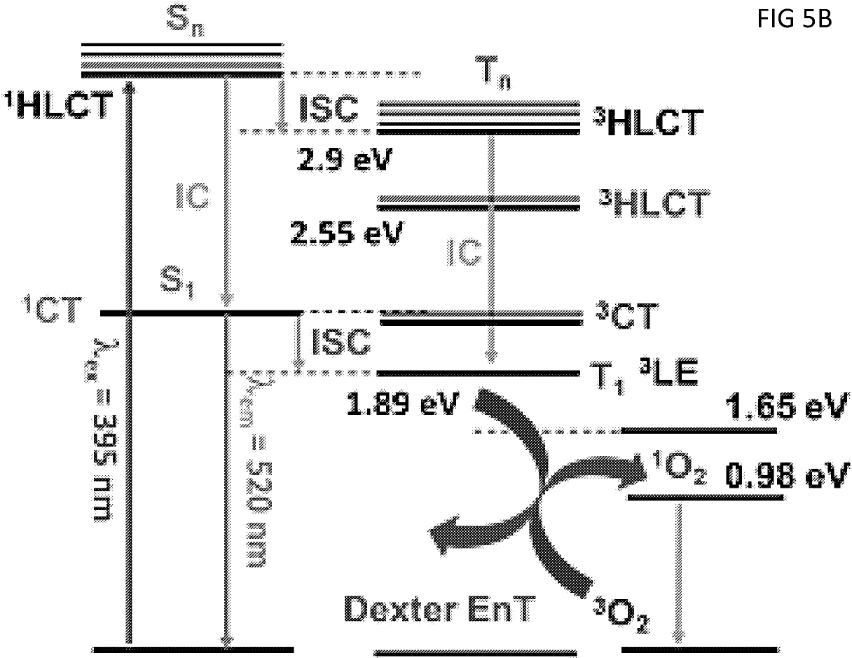

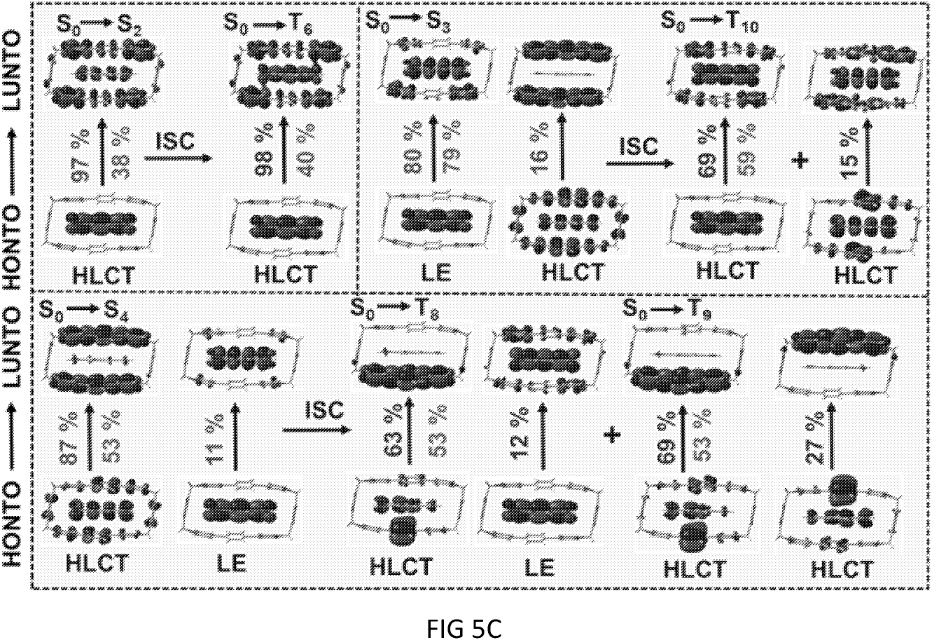

Nanosecond transient absorption measurements leads to the observation at λₑₓ = 414 nm of long-lived triplet of >1.5 µs, implying that excitation of the upper ¹CT and ¹LE states (S₂, S₃, S₄ states, vide infra) populates the T₁ state of the TBP following charge recombination, while excitation of the ¹CT states at 450 nm, does not lead to a detectable triplet population. The lack of triplet formation, following 450 nm excitation, is associated with the lower amount of triplet character in the CT state populated by absorption, which is also consistent with the discrepancy in the decay time constants at different excitation wavelengths. Whilst excitation at 414 nm offers shorter time-constants, associated with the more efficient SOCT-ISC between the upper states (S₂→T₆, S₃→T₆ and S₄→T₈ for example, FIG. 5A) with higher triplet character, excitation at 450 nm offers longer time-constants associated with slower SOCT-ISC between the S₁ and T₂ and T₃ states. Thus, the triplet population observed upon higher energy excitation (HLCT states) is a result of a rapid ISC induced by both the heavy Br atoms and SOCT-ISC between the D-A. These results are consistent with the efficient photocatalytic conversion of CEES at 395 nm, while photoexcitation at 450 nm the conversion of CEES is very slow (FIG. 15).

Solid-State Studies: Diffuse reflectance measurements on solid films of the ExBox•PSS composite exhibit (FIG. 3A) a broad peak in a range 200-450 nm centered on 350 nm. The ExBox•PSS emission is excitation-dependent, ranging from 470 nm to 525 nm (λₑₓ 380-450 nm). The emission is centered on 470 nm at λₑₓ = 380 nm, with a Stokes shift of 0.54 eV (Table 6) and singlet excited-state lifetimes (Φ_{F} = 1.32 %, τ₁= 0.22 ns, τ₂ = 1.40 ns, τ₃ = 8.23 ns at 298 K) (Table 2) slightly larger than those of in solution. The TBP⊂ExBox⁴⁺•PSS composites show (FIG. 3A) an intense broad reflectance peak in a range 200-440 nm (centered on 360 nm) and a small CT band at 455 nm similar to that of TBP⊂ExBox•4PF₆ in solution. The broad absorption spectrum extended up to 600 nm indicating the absence of a well-defined band edge in the UV-Vis energy range for all the materials. As in the case of TBP⊂ExBox•4PF₆ in solution, the composite TBP⊂ExBox•PSS displays (FIG. 3D) a first emission band at 440 nm and an exciplex emission band at 522 nm (λₑₓ = 380 nm, (Φ_{F} = 2.18 %) with a Stokes shift of 1.07 eV (Table 5-6), indicating of the persistence of the host-guest complex in the composite. The time-correlated emission measurements at λₑₓ = 405 nm revealed the existence of two components with different S₁ lifetimes at 440 nm (τ₁ = 0.14 ns, τ₂ = 1.32 ns, τ₃ = 4.03 ns) and 515 nm (τ₁ = 0.35 ns, τ₂ = 2.02 ns, τ₃ = 6.14 ns) (FIG. 3E, Table 1) which are associated to TBP and TBP⊂ExBox⁴⁺, respectively. It is noteworthy that the ¹S_{CT} lifetimes of TBP⊂ExBox⁴⁺ in both solution and the excited state are similar to the ¹LE states in the two components, TBP and ExBox•PSS, when they are separate from each other, indicating the existence of competing decay pathways. Furthermore, this observation indicates the formation of a new mixed dipole entity in TBP⊂ExBox⁴⁺ that exhibits faster relaxation from the CT state, consistent with the TA studies which support efficient ISC associated with the large SOC of the Br atoms but also the influence of SOCT-ISC in the D-A dyad.

Time-Dependent DFT (TD-DFT). In order to understand better the electronic properties of the TBP⊂ExBox⁴⁺ complex and have an estimation of the singlet-triplet energy gap (ΔE_{ST}), we utilized both the APFD and the B3LYP functionals in conjunction with the 6-31G(d) basis set to calculate molecular geometries. Optimization of the superstructure of the TBP⊂ExBox⁴⁺ at the B3LYP/6-31G(d) energy level leads to a larger interplanar distance between the TBP and the Exbipy²⁺ units (~4.2 Å), while utilization of the APFD functional offers a superstructure with interplanar distances between the TBP and Exbipy²⁺ of 3.5 Å, similar to those reported²² from the crystals structures of polyaromatic compounds inside the ExBox⁴⁺. The discrepancy between these optimized superstructures is a result of incorporation of an empirical dispersion correction term within the APFD formalism, while dispersion interactions are neglected within the B3LYP fuctional.³¹ In addition, these two geometries offer the possibility to determine the energy of the LE states of the TBP and ExBox⁴⁺ and, hence, unravel the role of the orbital overlap between the D-A into the formation of mixed excited states. Satisfied by the presence of zero negative frequencies, gas-phase TD-DFT calculations have been subsequently, carried out at the B3LYP/6-31G(d) level of theory using Gaussian16 software.³² Here we discuss the electronic properties of TBP⊂ExBox⁴⁺ derived from the APFD/6-31G(d) optimized structure (FIG. 5, Table 2), while a detailed analysis of the electronic properties of the B3LYP/6-31G(d) geometry can be found in the Supplementary Information. The molecular electrostatic potential difference map (CI - SCF) of the TBP⊂ExBox⁴⁺ complex revealed that the negative electron density is localized on TBP, while the positive charge density is localized on ExBox⁴⁺, consistent with the electron D-A nature of the complex. The calculated absorption spectrum reproduces well the experimental absorption profile with dominant bands at 420 and 387 nm. As expected, the HOMO is localized on TBP while the LUMO is localized on ExBox⁴⁺ with an ΔE_{H-L} (HOMO-LUMO energy gap) of 2.74 eV (452 nm) (Table 5).

The singlet and triplet excited states of the TBP⊂ExBox⁴⁺ complex consist of (FIG. 5A, Table 3) LE states in the TBP or ExBox⁴⁺ components, CT excited states, and hybridized locally charge transfer excited state (HLCT) which is a mixed state between the LE and CT states.³² The first CT transition (Table 3) which is the S₀→S₁ transition (552 nm, ƒ = 0.0004), is associated essentially with the HOMO→LUMO+1 (99%) transition. The energy of the S₁ state is consistent with the observation of exciplex emission at 520 nm associated with the radiative charge recombination in the D-A dyad. The S₀→S₂ transition at 2.99 eV (415 nm, ƒ= 0.0097) corresponds to the ¹HLCT state in involving a minor ¹LE transitions on the TBP (HOMO-LUMO+2 (34%)) and a major ¹CT components (HOMO→LUMO+3, 63%) (Table 3). These results are consistent with the weak broad absorption band observed experimentally at 455 nm. FIG. 5A presents the excited state energy diagram and transition configurations of a singlet (Sₙ) and triplet (Tₙ) excited states of TBP⊂ExBox⁴⁺. Previous investigations have revealed⁹ that the S-T transformation is rather facile when the two excited states contain the same components of transition configurations to establish the transformation channels in bridging the spin-forbidden transitions between two electronic states with different spin multiplicities. The S_{0→}S₁ transition possesses a very weak oscillator strength and involves only a CT transitions from TBP to ExBox⁴⁺. Notably, the lowest triplet state (T₁, 1.89 eV) contains two components—namely, TBP→TBP (HOMO-LUMO+2, 88%) and CT (HOMO→LUMO+3, 5%) relaxation processes, which can be considered essentially as a ³LE state. Computed singlet and triplet excited states of only TBP revealed that the T₁ (1.91 eV) state has similar energies as those found for TBP⊂ExBox⁴⁺, and is significantly lower in energy than the S₁ and T₂ states hampering, therefore, its population either through ISC or IC excited states relaxation mechanisms. The S₀→T₃ (2.24 eV) transition is identical to the S_{0→}S₁ transition and is associated with a CT transitions in the TBP⊂ExBox⁴⁺ host-guest complex. The S-T transformation between the S₁ and T₃ states occurs through the SOCT-ISC (Table 2) since the ΔE_{ST}¹³ = 0.0076 eV (<<0.37 ev). The extent of the HOMO to LUMO orbital overlap is small (15%) consistent with the interplanar distances between the TBP and Exbipy²⁺ units, of ~3.5 Å, similar to Van der Waals radii (3.5 Å) between carbon atoms. These distances are similar to the those reported²² from crystal structures of the Pyrene⊂ExBox⁴⁺. It was previously proposed⁹ that the minimum requirement for realizing exciton transformation is the matching of the energy levels of the two states based on the thermal equilibrium between the singlet and triplet excited states. Although the exciton transformation channel S₁→T₃ (¹CT→³CT) has a very small ΔE_{ST} (~ 0 eV), the weak ƒ of the CT transitions in the TBP⊂ExBox⁴⁺ leads to a low population of the T₁ state as observed by TA experiments, offering (FIG. 69), therefore, a weak photosensitizing efficiency at λₑₓ = 450 nm.

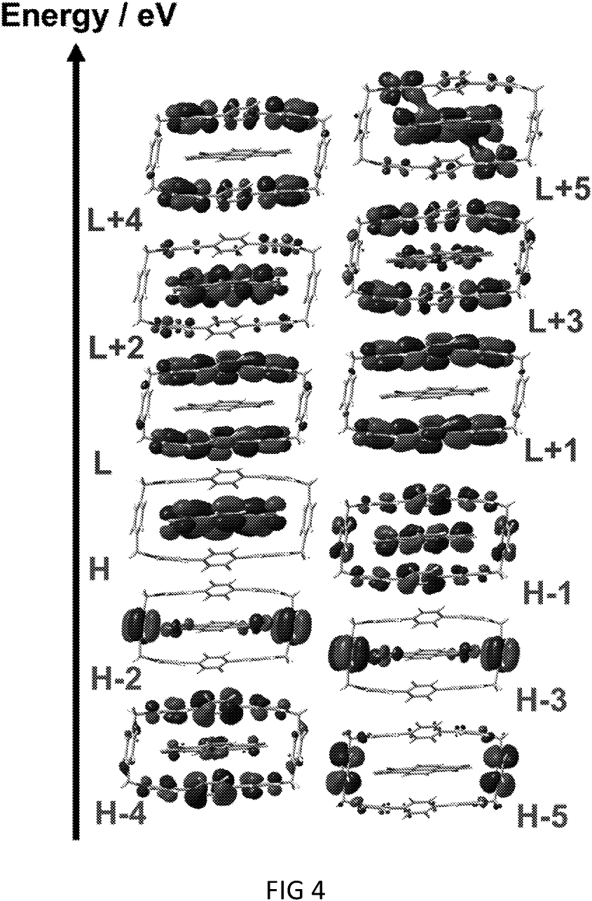

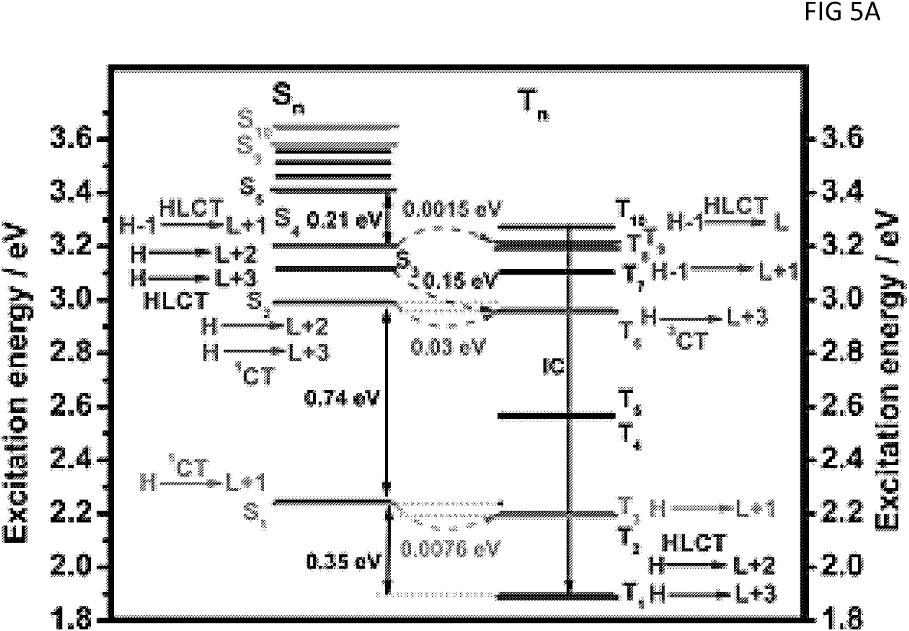

The photocatalytic performance of the TBP⊂ExBox⁴⁺ D-A dyad is high in the excitation range 380-420 nm (λₘₐₓ = 395 nm, 3.13 eV) and so, the photocatalytic properties arise from the ¹HLCT states, S₂, S₃ and S₄ states (band at 387 nm, FIG. 48B). The S₀→S₂ (2.99 eV) and S₀→S₃ (3.11 eV) and S₀→T₁ (1.89 eV) transitions are similar (HOMO→LUMO+2 and HUMO→LUMO+3), and the ΔE_{ST} is very large (> 1 eV), hampering (FIG. 5A) efficient ISC between S₂→T₁ and S₃→T₁ channels. From the TA experiments, the S-T transformation is more efficient when higher energy excited states are accessed. The S₀→T₆ transition configuration is very similar to that of S₀→S₂ and S₀→S₃, containing both a high HOMO→LUMO+3 component. The ΔE_{ST} between the S₂ and S₃ states with the T₆ states is very small (ΔE_{ST}²⁶ = 0.03 and ΔE_{ST}³⁶ = 0.15 eV respectively) and implies a facile exciton transformation. It is noteworthy that the S₀→S₄ state is characterized by a large oscillator strength (ƒ= 0.24) and arises (FIG. 4, Table 2) predominantly the HOMO-1→HUMO+1 transition. From Table 3, the S₄→T₈ and S₄→T₉ ΔE_{ST} is very small and can serve as potential channels for ISC. Previous investigations have shown that, even in the absence of heavy atoms, CT states can undergo efficient ISC through either radical-pair intersystem crossing³⁴ (RP-ISC) for long-lived charge separated states or recombination to form local triplet excited states of either the D or A units using SOCT-ISC.¹⁹ Recently, dyads combining BODIPY as an electron acceptor and pyrene or perylene as electron donor subunits were shown to display⁷ CT states formed as a result of photoinduced electron transfer and were found to yield triplet excited states of the BODIPY.

In order to decipher further the contribution of the LE, CT and HLCT states to the overall ISC process in the TBP⊂ExBox⁴⁺ complex, natural transition orbital (NTO) analysis, based on the singular value decomposition of 1-particle transition density matrix, was performed. NTOs give a compact representation of the orbital transformation composition for a given transition. The highest occupied natural transition orbital (HONTO) and the lowest unoccupied natural transition orbital (LUNTO) orbitals represent any one electron property associated with the electronic transition and excitation amplitude is always the most significant for any particular excited state, as a result of its dominating role in determining the one electronic transition for the generation of the corresponding excited state from the ground state (S₀).³⁵ The HONTOs and LUNTOs of all the hybridized singlet (S₂, S₃ and S₄) and triplet states (T₆, Ts, T₉, and T₁₀) were investigated. Within the singlet/triplet excited state pairs that can undergo exciton transformation (FIG. 5C) according to the energy gap law (|ΔE_{ST}| < 0.37 eV), very similar HONTO and LUNTO distributions at both singlet and triplet excited states were observed in TBP⊂ExBox⁴⁺ where the D dominates HONTO and the A determines LUNTO for very small overlap between HONTO and LUNTO. The almost identical HONTO and LUNTO distributions for S₀ →S₂ (Is = 37%) and S₀ → T₆ (I_{T} = 40%) transitions, and the small ΔE_{ST} (<0.37 eV) combined with non-negligible orbital overlap, provide a facile exciton transformation channel for efficient ISC processes between S₂ and T₆. The NTOs of the S₃ and T₁₀ excited states revealed that they have predominantly a LE character associated with the TBP, with a small contribution from the CT states, supporting the role of both the Br atom and CT for in the overall ISC process. The S₄→T₈ and S₄→T₉ channels are associated with a CT TBP to the ExBox⁴⁺ with a small contribution from the ExBox⁴⁺↔ExBox⁴⁺ ISC. At higher excitation energy (>3.4 eV), CT from the p-xylylene unit of the Exbox⁴⁺ is triggered as reported¹³ previously. In this context, utilization of D-A complexes, showing HLCT character (FIG. 5B) can shed important light on the fundamental S-T exciton transformation mechanism in host-guest supramolecular organic complexes, stimulating further the research into purely organic materials capable of facile exciton transformation.

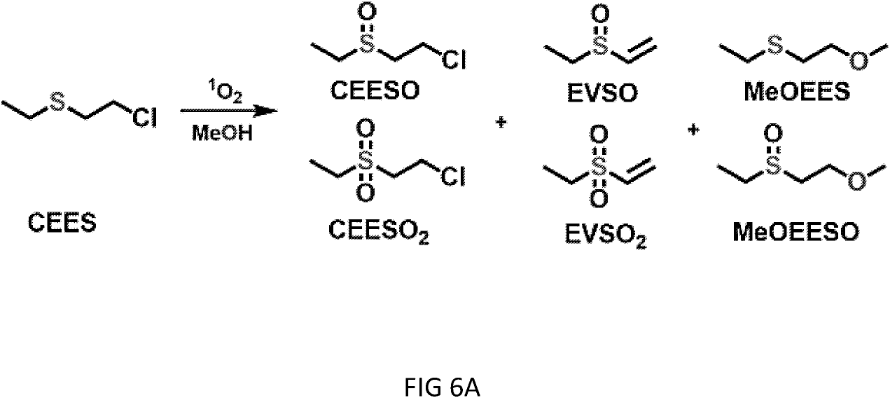

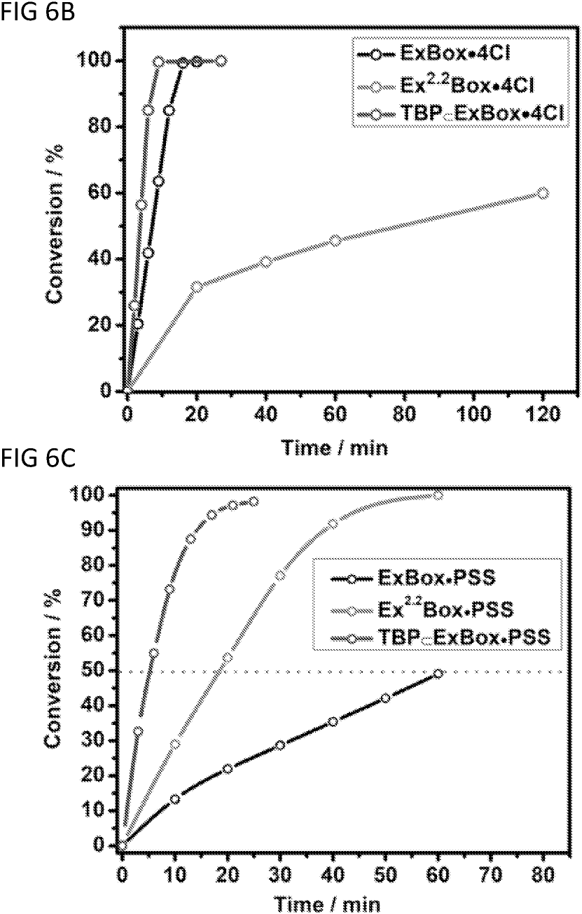

Photocatalytic Activity. Generation of ¹O₂ by stable microporous organic photocatalysts in both aqueous and organic media provide countless opportunities, not only for the development of environmentally and economically viable materials for the elimination of SM stockpiles, but also in the design MPEs. Compared to other oxidants, the reaction of ¹O₂ with CEES leads to the selective formation of less toxic CEESO as a major product, while CEESO₂ is formed as a minor product (FIG. 6A) in MeOH solution.^{10f} In this study, the photocatalytic activity of the supramolecular photosensitizer TBP⊂ExBox⁴⁺ has been explored in both the homogenous and heterogeneous media. In order to confirm the role of the exciton transformation in the D-A dyad, we also investigated the photocatalytic performances also of the TBP, ExBox⁴⁺ and Na•PSS separately in order to unravel the contribution of each components to the overall catalytic activity of the composites. In addition, we explored the photocatalytic activity of the light sensitive Ex^{2.2}Box⁴⁺ in order to emphasize the role of incorporating tetracationic cyclophanes into a PSS matrix so as to increase the photostability, resulting in the enhancement of the photocatalytic selectivity. All the catalytic results are summarized in Table 3 and 4.

Homogenous Photocatalysis. The photocatalysis of CEES with 1% mol catalyst of ExBox•4Cl, Ex^{2.2}Box•4Cl or TBP⊂ExBox•4Cl has been carried out (FIG. 6B and FIG. 56) in CD₃OD under photoirradiation at 395 nm. The kinetics of the conversion of CEES has been monitored by ¹H NMR and decoupled ¹³C NMR. The ExBox•4Cl photocatalyst led (FIG. 6B) to a 100% conversion of CEES to the CEESO after 16 min irradiation. The half-life time of the reaction was observed to be 7 min. The ¹H NMR spectrum of CEES contains two triplets centered on 3.7 and 2.9 ppm. After irradiation, these peaks disappear and two multiples appear at 4.0 and 3.3 ppm, corresponding to the chemical shifts of CEESO. Formation of this sulfoxide product was also confirmed by ¹³C NMR spectroscopy. After 20 min photoirradiation, ¹H NMR spectroscopy shows the formation of the 2% CEESO₂ based on the appearance of a peak at 3.57 ppm and negligible amount of vinyl derivatives. We tested also the photoactivity of the Ex^{2.2}Box•4Cl as a photosensitizer for the oxidization of CEES, however, we found out that only 45% of CEES was converted in 60 min by Ex^{2.2}Box•4Cl.It is noteworthy, the Ex^{2.2} Box•4Cl leads to the formation of different major products, such as CEESO and MeOEES and MeOEESO (FIG. 6A, FIG. 13). MeOH can stabilize sulfide-sulfoxide intermediate³⁶ while CEES can undergo methanolysis and form MeOEES if CEES is exposed to MeOH for long period of time. The low selectivity —only 34% of CEESO formed after 120 min, Table 3— of the Ex^{2.2}Box•4Cl is associated with the decomposition of the Ex^{2.2}Box⁴⁺ under photoirradiation as it is confirmed by the UV-Vis absorption and ¹H NMR spectroscopies. Since TBP is insoluble in MeOH, its photocatalytic performances in homogenous media cannot be obtained. The TBP⊂ExBox•4Cl D-A dyad is soluble in MeOH and leads to significant increase in CEES oxidation rate (FIG. 6B, Table 3) reaching a 100% conversion in less than 10 min (t_{½} = 3.5 min). Compared to ExBox⁴⁺, inclusion of the TBP significantly enhances the CEES conversion rate. In addition, ¹H and ¹³C NMR spectra of photooxidation processes catalyzed by TBP⊂ExBox⁴⁺•4Cl showed full conversion of CEES to its sulfoxide form with no detectable toxic sulfone formation.

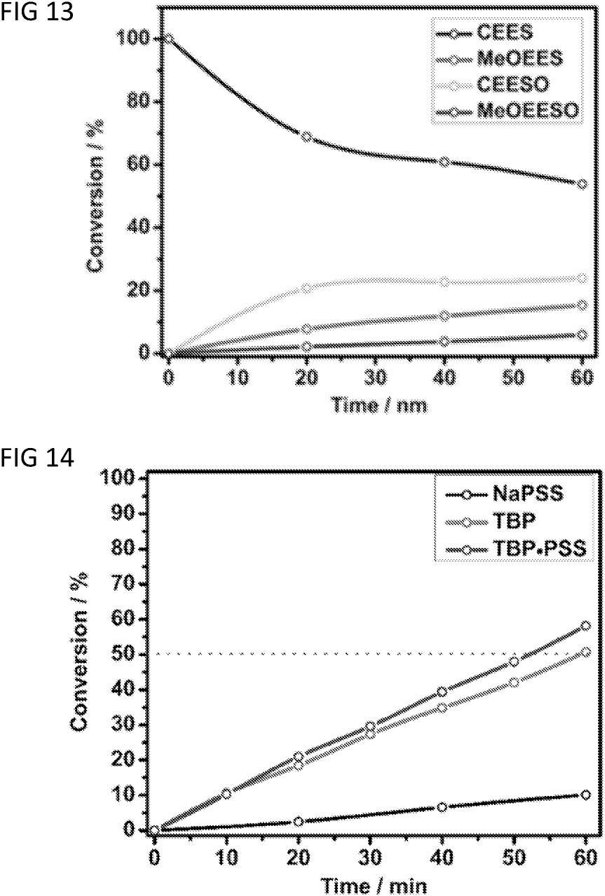

Heterogenous Photocatalysis. The design of protective equipment against chemical warfare agents such as SM requires the development of efficient heterogeneous photocatalyst. The fulfilment of this goal requires taking into account multiple parameters namely— (i) the polymer matrix needs to be transparent in order for the photocatalyst to be able to absorb a maximum of light irradiation, (ii) the polymer is porous to allow a facile transport of species to and from the active sites, (iii) the photosensitizer needs to be photostable in relation to photobleaching, (iv) the stabilization of specific transition state is required in order to optimize the selectivity, and finally (v) the different components need to be insoluble to avoid chemical leaching. Blending cationic cyclophanes with commercially available an anionic polymer matrix leads to the formation of insoluble composites with relatively large surface areas, a characteristic that can help the transport of reactant (³O₂, CEES) and products (¹O₂ and CEESO) to and from the photocatalytic sites. The heterogenous catalytic reactions have been achieved with 1 mol% catalyst of ExBox•PSS, Ex^{2.2}Box•PSS, and TBP⊂ExBox•PSS under photoirradiation at 395 nm (FIG. 6C). Control reactions have been conducted with PSS and TBP•PSS composites and TBP using identical amount of the catalyst (1 mol%) to estimate the contribution of the PSS and TBP components into the overall photocatalytic performance (FIG. 14). All data are summarized in the Table 4.

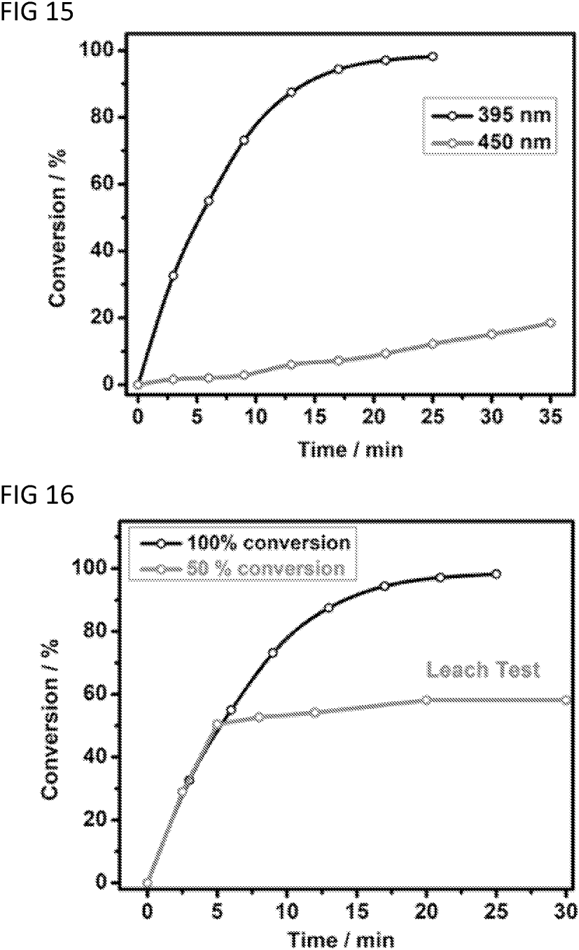

The Na•PSS did not show significant photocatalytic performance except for a slight conversion because of the decomposition of CEES to MeOEES in MeOH (FIG. 6A). The CEES conversion is significantly slower with the ExBox•PSS in heterogenous media (FIG. 6C, Table 4) compared to the photocatalytic performance of the ExBox•4Cl in homogenous media. After 60 min of photoirradiation, the conversion of CEES did not exceed 40%. ¹H and ¹³C NMR spectra revealed the selective formation of CEESO. The slow photoactivity of the ExBox⁴⁺ is consistent with the longer S₁ lifetime (~8 ns) in solid state than in solution (~1.4 ns) indicating the less efficient ISC in the solid state in the ExBox⁴⁺. As in the case of ExBox•PSS, the TBP-Na•PSS composite showed (FIG. 14) less than 50% conversion of CEES, implying the low ¹O₂ generation. This observation is consistent with DFT calculations which have shown that the T₁ state of TBP cannot be populated efficiently through an ISC or IC mechanism because of the large energy barriers. Surprisingly, Ex^{2.2}Box•PSS composite showed a catalytic performance in heterogenous media with 100% in 60 min and t_{½}= 18 min, whilst in homogenous media, the conversion did not exceed 45% within a similar photoirradiation time-frame. More importantly, the formation of CEESO was fully selective and ¹³C NMR spectroscopy did not show the appearance of the MeOEES, as observed in homogeneous catalysis as a result of the enhanced stability of the Ex^{2.2}Box⁴⁺ cyclophane within the PSS matrix. Previous investigations³⁷ have shown the increase of the stability of air-sensitive S/N radicals when incorporated polymers matrices. The TBP⊂ExBox•PSS composite have registred a 12-fold increase in the kinetic of the conversion of CEES to CEESO comparing to ExBox·PSS and TBP-Na•PSS at λₑₓ = 395 nm (FIG. 6C, FIG. 14). Within 20 min of photoirradiation, the conversion reached 100% with t_{½} = 5 min and 100% selectivity for the formation (Table 4) of CEESO. These results are consistent with DFT calculations and spectroscopic investigations showing that excitation of the HLCT excited states triggers an efficient S-T transformation that leads to the population of the T₁ state. The energy of the T₁ state is of 1.89 eV energy and is close to the first excited state of the ¹O₂ which is of 1.63 eV,²⁰ offering (FIG. 5B), therefore, an energy gap of ~0.25 eV which ideal for efficient Dexter energy transfer. ^{18,20} Noteworthy, photocatalysis of CEES at λₑₓ = 450 nm is very slow, confirming the weak population of the T₁ states upon excitation of the low energy ¹CT states. In order to confirm the stability of the TBP⊂ExBox•PSS photocatalyst, we performed a leaching test which consists (FIG. 16) of the removal of the catalyst upon reaching 50% conversion of CEES. Further irradiation of the solution has shown no significant change in the concentration of CEES indicating that (i) the TBP⊂ExBox•PSS composite is responsible for the transformation of the CEES to CEESO under photoirradiation and (ii) the composite is stable under the photocatalytic conditions because of the absence of chromophores in solutions. Further photoirradiation does not lead to a change in the CEES conversion confirming the stability of the supramolecular TBP⊂ExBox•PSS photocatalyst.

Supramolecular porous organic composites based of tetracationic cyclophanes (ExBox⁴⁺ and Ex^{2.2}box⁴⁺) and an anionic polymer matrix such as polystyrene sulfonate (PSS) have been prepared. These materials were found to be microporous as evidenced by CO₂ adsorption isotherms. In addition, larger molecules such as TTF can diffuse inside the polymer confirming the possibility for larger molecules to diffuse in/out of the ExBox•PSS composite. While the photocatalysis of CEES by ExBox•4Cl in solution is fast and selective, in the solid state the conversion of the CEES to CEESO is very slow as the result of stabilization of the singlet excited state. Other cyclophanes, such as Ex^{2.2}Box•4Cl are not stable under photoirradiation and the photocatalysis of CEES is slow and not selective. Notably, Ex^{2.2}Box•PSS is stable under photoirradiation and the conversion of CEES to CEESO is 100% selective. Although the lowest triplet state (T₁) of the 1,3,5,8-tetrabropyrene (TBP) is very low in energy, it is inaccessible on account of the large energy barrier separating the T₁ states from the S₁ and T₂ states. The efficiency of the singlet to triplet (S-T) transformation in the TBP⊂ExBox⁴⁺ host-guest complex is associated with a combination of both a large spin-orbit coupling of the Br atoms, with spin-orbit charge transfer intersystem crossing of the D-A dyad. In addition, DFT calculations revealed the existence of a manifold of excited states that can enhance the internal conversion (IC) of the upper triplet states to populate the low lying T₁ excited state. This efficient S-T transformation and IC played a central role in the enhancement of the ¹O₂ generation and subsequently increase in the photocatalytic performances. The high stability, facile preparation, processability and high performance of the TBP⊂ExBox•PSS composite augur well the future development of the supramolecular heterogenous photosensitizer using host-guest chemistry. More broadly, these results reveal a number of other opportunities for facile fine-tuning the S-T transformation in D-A dyads using host-guest chemistry which can unleash several fundamental and technological advances for future design of triplet excited state chromophores.

### Materials / General Methods / Instrumentation

All chemicals and reagents were purchased from commercial suppliers (Aldrich and TCI chemicals) and used without further purification. Exbox•4PF₆, ExBox•4Cl and Ex^{2.2}Box•4PF₆ were prepared according to previous literature procedures.¹ Column chromatography was carried out on silica gel 60F (Merck 9385, 0.040-0.063 mm). ¹H and ¹³C Nuclear magnetic resonance (¹H and ¹³C NMR) spectra were recorded on a Bruker Avance 500 with working frequencies of 500 MHz. Chemical shifts are reported in ppm relative to the signals corresponding to the residual non-deuterated solvents (CD₃CN: δ = 1.94 ppm, D₂O: δ = 4.79, CD₃OD δ= 3.34). Gas Chromatography GC-FID measurements were carried out on an Agilent Technologies 7820A GC system equipped with an Agilent J&W GC HP-5 capillary column (30 m × 320 µm × 0.25 µm film thickness). Heterogenous samples were filtered and diluted with CH₂Cl₂ prior to injection. Starting temperature: 70° C., Hold: 0.5 min, Ramp: 30° C./min, Time: 1 min, Ramp: 75° C./min, End temperature: 250° C. The disappearance of the reactant was calculated relative to a 0 min time point.

### Preparation Protocols

### A. Preparation of ExBox·PSS Composite

a) ExBox·PSS with 1/1 w/w ratio. ExBox•4Cl (35 mg, 0.043 mmol) and sodium polystyrene sulfinate (Na-PSS) (36 mg, 0.172 mmol) were dissolved separately in H₂O (5 ml). The number of moles is determined according to the repeating unit (C₈H₇SO₃Na) of molecular weight of 206.19 g.mol⁻¹. Therefore, to achieve a full exchange of the Cl⁻ anions of the ExBox•4C1, four equiv of the (C₈H₇SO₃Na) are required.The amount of Na·PSS utilized was calculated according to the number negative charges. Therefore, four equivalents of Na·PSS unit are needed to exchange the four chloride ions of the Exbox•4Cl. Upon dropwise addition of Na·PSS into an aqueous solution of the ExBox•4Cl, a light-yellow pale precipitate is formed immediately. After stirring the mixture for 1 h, the solid was isolated by centrifugation and was washed three times with H₂O to remove the NaCl. Yield: 55 mg.

b) ExBox·PSS with ⅓ w/w ratio. ExBox•4Cl (32 mg, 0.04 mmol) have been solubilized in MeCN (5ml) while sodium polystyrene sulfinate (Na•PSS) (98 mg, 0.47 mmol) have been solubilized in H₂O (5 ml). To increase the solubility of the composite in water, we utilized 12 equiv of the (C₈H₇SO₃Na) unit of the Na·PSS polymer. Upon dropwise addition of an aqueous solution of Na·PSS into an aqueous solution of the ExBox•4Cl, a light-yellow pale precipitate is formed immediately. After stirring the mixture for 1 h, the solid was isolated by centrifugation and the solid was washed three times with H₂O to remove NaCl. Yield: 60 mg.

B. Preparation of Ex^{2.2}Box•PSS composite at 3/2 w/w ratio: Ex^{2.2}Box•4PF₆ (30 mg, 0.022 mmol) was solubilized in MeCN (5 mL) to afford a pale-yellow solution. Sodium polystyrene sulfinate (Na-PSS) (19 mg, 0.091 mmol) was solubilized in H₂O (5 mL). The number of moles of PSS utilized corresponds to the number of negative charges required to exchange all the PF₆ counterions of the Ex^{2.2}box⁴⁺. Upon dropwise addition of the solution of the Na·PSS into the solution of Ex^{2.2}Box•4PF₆, a dark yellow precipitate formed immediately. After stirring the mixture for 1 h, the solid was isolated by centrifugation and washed three times with H₂O to remove the Na•PF₆. Yield: 28 mg, 77%. The number of moles is determined according to the repeating unit (C₈H₇SO₃Na) of molecular weight of 206.19 g.mol⁻¹. Therefore, to achieve a full exchange of the (PF₆)⁻ anions of the Ex^{2.2}Box•4PF₆, four equiv of the (C₈H₇SO₃Na) are required.

### C. Preparation of Ex^{2.2}Box•PSS Composite at 1/1 w/w ratio:

Ex^{2.2}Box•4PF₆ (17 mg, 0.012 mmol) was solubilized in MeCN (5ml) to afford a pale-yellow solution. Sodium polystyrene sulfinate (Na•PSS) (18 mg, 0.092 mmol) was solubilized in 5 ml of water. The number of moles of PSS utilized corresponds to the number of negative charges required to exchange all the PF₆ counterions of the Ex^{2.2}box⁴⁺ Upon dropwise addition of the solution of the Na·PSS into the solution of the Ex^{2.2}Box•4PF₆, a bright yellow colored solution formed immediately in an excess of H₂O. After stirring the mixture for one hour, the solvent was evaporated and the isolated solid was washed three times with acetonitrile to remove the Na•PF₆. The number of moles is determined according to the repeating unit (C₈H₇SO₃Na) of molecular weight of 206.19 g.mol⁻¹. Therefore, to achieve a full exchange of the (PF₆)⁻ anions of the Ex^{2.2}Box•4PF₆, four equiv of the (C₈H₇SO₃Na) are required. Yield: 19 mg.

### D. Preparation of TBP⊂ExBox•4PF₆ Complex (Scheme 4)

ExBox•4PF₆ (30 mg, 0.024 mmol) was solubilized in dimethylformamide (5 mL) to afford a colorless solution. Excess of 1,3,6,8-tetrabromopyrene (TBP) (37 mg, 0.071 mmol) was dissolved in hot PhMe to afford a pale yellowish solution which was added dropwise to the solution of ExBox•4PF₆ at 80° C. The mixture was kept warmed at 80° C. for 24 h leading to the evaporation of the PhMe and offering a dark yellowish solution of TBP⊂ExBox•4PF₆ in DMF. After complete evaporation of the solvent, a crude yellow powder of TBP⊂ExBox•4PF₆ contaminated with an excess of TBP was isolated. MeCN was added to the crude product in order to solubilize the TBP⊂ExBox•4PF₆ complex and remove the insoluble excess of TBP by filtration. After drying the yellow solution, TBP⊂ExBox•4PF₆ was isolated as a bright yellow powder. Yield: 40 mg, 94%.

Scheme 4: Preparation of the TBP⊂ExBox•4PF₆ complex.

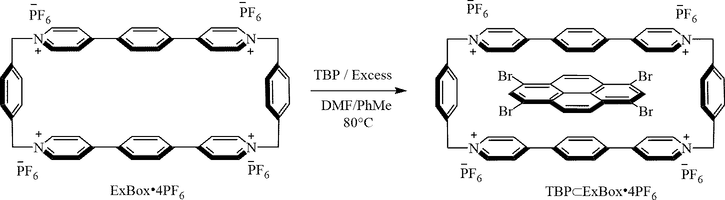

### A. Preparation of TBP⊂ExBox•4Cl Complex (Scheme 5)

TBP⊂ExBox•4PF₆ (20 mg, 1.5 × 10⁻⁵ mmol) was dissolved in MeCN (5 mL). Tetrabutylammonium chloride (50 mg, 0.18 mmol) is added to exchange the PF₆ anions with chloride anions. After centrifugation and several washes with MeCN, a yellow powder was obtained which was dried under vacuum for 24 h. Yield: 12 mg, 80%.

Scheme 5: Preparation of ExBox•4Cl composite

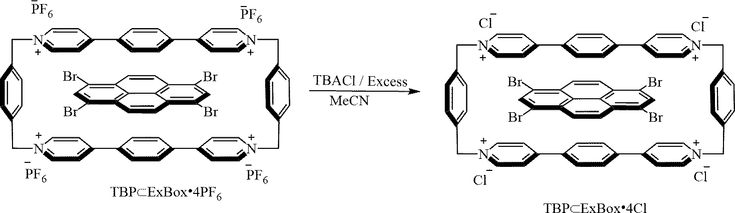

### A. Preparation of TBP⊂ExBox•PSS Complex (Scheme 6)

TBP⊂ExBox•4Cl (10 mg, 0.0075 mmol) and Na·PSS (6 mg, 0.031 mmol) were dissolved separately in H₂O (5 mL). The amount of Na·PSS utilized was calculated according to the number negative charges.The number of moles is determined according to the repeating unit (C₈H₇SO₃Na) of molecular weight of 206.19 g.mol⁻¹. Therefore, to achieve a full exchange of the Cl⁻ anions of the TBP⊂ExBox•4Cl, 4 equiv of the (C₈H₇SO₃Na) are needed. Thus, 4 equiv of sodium styrene sulfonate units are required to fully exchange the 4 Cl⁻ atoms of the TBP⊂ExBox•4Cl. Upon addition dropwise of the solution of the Na·PSS into the solution of the TBP⊂ExBox•4Cl, a bright yellow pale precipitate of TBP⊂ExBox•PSS is formed immediately. After stirring the mixture for 1 h, the solid was isolated by centrifugation and washed two times with H₂O. Yield: 11 mg, 73%.

Scheme 6: Preparation of TBP⊂ExBox•PSS composite

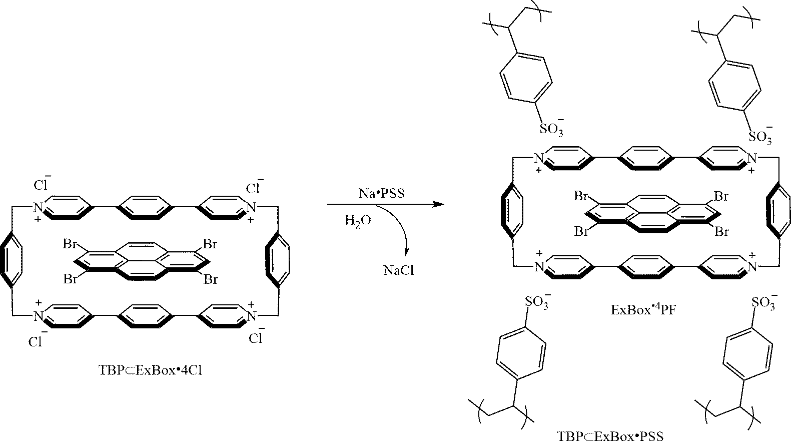

### Powder X-Ray Crystallography Characterization

Powder X-ray diffractions were conducted on a STOE-STADI MP powder diffractometer equipped with an asymmetric curved Germanium monochromator (CuK_{α1} radiation, λ = 1.54056 Å) and a one-dimension silicon strip detector (MYTHEN2 1 K from DECTRIS). The line focused Cu X-ray tube was operated at 40 kV and 40 mA. Samples for structural analysis were measured at room temperature in transmission geometry.

### Gas Adsorption Studies

Adsorption Isotherms. The CO₂ adsorption isotherms of ExBox·PSS were measured at 278 K and 195 K using a Micromeritics ASAP 2020 instrument. Pore-size distributions were estimated using 2D-NLDFT (N₂-carbon finite pores, As = 6) method with a non-negative regularization of zero. The CO₂ adsorption isotherms of Na·PSS and Ex^{2.2}Box•PSS were measured at 195 K using a Micromeritics ASAP 2020 instrument. The activation of Na·PSS, ExBox·PSS and Ex^{2.2}Box•PSS was achieved by a supercritical-drying process using a TousimisTM Samdri® PVT-3D critical point dryer (Tousimis, Rockville, MD, USA) in which liquid CO₂ was used to exchange the CO₂ five times over the course of 10 h. The materials were heated to above the critical point of CO₂ (T = 31° C., P = 73 atm) and the instrument was bled at a rate of ~0.5 sccm. Finally, samples were degassed at 35° C. for 6 h under high vacuum on a Smart Vacprep from Micromeritics. Around 30-50 mg of sample was used in each measurement, and the BET surface areas were calculated in the region P/Pₒ = 0.005-0.05.

### Scanning Electron Microscope

The SEM images and map scans were collected on a Hitachi SU8030 FE-SEM (Dallas, TX) microscope at Northwestern University’s S-9 EPIC/NUANCE facility. Samples were activated and coated with O_{S}O₄ to ~ 9 nm thickness in a Denton Desk III TSC Sputter Coater (Moorestown, NJ) before imaging.

### Optical Spectroscopy Characterization

Solution UV/Vis absorption spectra were recorded using a UV-3600plus Shimadzu spectrophotometer. The fluorescence spectra are collected using the Horiba Fluoromax-4 Spectrophotometer. Preparation of thin films for solid-state investigations was carried out by drop-casting on quartz slide. After solvent evaporation, thin films are formed.

Diffused reflectance spectra for the solid samples were measured using a JASCO V-670 UV-Vis-NIR Spectrophotometer equipped with a 60 mm BaSO₄-coated integrating sphere and a PMT//PbS detector. Steady-state emission and excitation-emission mapping spectra were recorded at room temp using an Edinburgh Instruments FS5 spectrofluorimeter. Samples for spectroscopic measurements were packed inside a quartz capillary tube (ID = 3 mm), charged with degassed MeTHF solvent, and then sealed inside the glovebox: the samples were then soaked overnight. The spectra were collected in the front-face configuration using a 1.4 nm excitation and 0.4 nm emission slit widths and corrected by using the instrumental correction functions for the excitation light source as well as detector response. The absolute quantum yields (QYs) were measured using a 150 mm integrating sphere. QY values were calculated with EI F980 software that accounts for the diminished intensity (photon counts) of the incident excitation beam over the increased intensity (photon counts) of fluorescence, based on the manually selected respective integration range. Fluorescence lifetime emission decay profiles were recorded using an Edinburgh Lifespec II Picosecond Time-Correlated Single Photon Counting Spectrophotometer equipped with a Hamamatsu H10720-01 detector and a 405 nm picosecond pulsed diode laser as TCSPC source (IRF ≈180 ps). An iterative deconvolution procedure with exponential fitting was used within the EI F980 software to extract lifetime data.

The excitation-wavelength dependent fluorescence (Red-edge effect phenomena)² is related to a slow solvation dynamic in relation to the time scale of the fluorescence.

### Transient Absorption Spectroscopy

The setup for transient absorption measurements has been described elsewhere.³ Photoexcitation pulses at 414 nm were obtained through a β-barium borate (BBO) crystal doubling the fundamental, and the 450 nm pulses were generated with a commercial non-collinear optical parametric amplifier (TOPAS-White, Light-Conversion, LLC). The pulse energy for photoexcitation was attenuated to ~1 µJ/pulse using neutral density filters. The pump polarization was randomized employing a commercial depolarizer (DPU-25-A, Thorlabs, Inc.) to eliminate any orientational dynamics contributions from the experiment. All the spectra were collected on a commercial spectrometer (Ultrafast Systems, LLC Helios and EOS spectrometers, for fsTA and nsTA, respectively). All samples were stirred to avoid localized heating or degradation effects. The optical density was maintained around 0.5 for all samples.

### Optical Properties of Ex^{2.2}Box•PSS

The Ex^{2.2}Box•PSS has yellow pale color in H₂O and the UV-vis absorption profile is characterized by the existence of two broad absorption bands at 360 and 390 nm, while fluorescence spectroscopy has shown that excitation at 380 nm offers a single emission band at 430 nm with Φ_{F} = 35% (λₑₘ = 430 nm, τ₁ = 0.31 ns, τ₂ = 1.23 ns). In the solid state, although excitation at different wavelengths (390 and 414 nm) offers a similar emission broad band at 490 nm), the singlet excited state display excitation-dependence behavior similar to the ExBox·PSS composite.

### Photostability of Ex^{2.2}Box•4PF₆ in MeOH un Photoirradiation at 395 nm

The photostability of the Ex^{2.2}Box•4PF₆ was monitored using absorption and ¹H NMR spectroscopies under photoirradiation at 395 nm in MeOH. After 1 h irradiation, both the UV-vis absorption and ¹H NMR spectra undergo significant changes indicating the decomposition of the Ex^{2.2}Box•4PF₆.

### Section G. TD-DFT Calculations

### TD-DFT Calculation on 1,3,6,8-Tetrabromopyrene (TBP)

The structure of 1,3,6,8-tetrabromopyrene (TBP) was optimized at the B3LYP/6-31G(d) level. Time-dependent DFT (TD-DFT) calculations were carried out on the singlet (S) and triplet (T) states on the optimized gas-phase geometry using Gaussian 16 package,⁴ considering a total of 20 excited states. Three singlet state transitions were determined with oscillator strengths (ƒ) > 0.05 and these are tabulated in Table 8. The energy levels of the S and T states possess a possible intersystem crossing channel between the S₁ and T₂ state. The T₁ state is very low in energy (1.91 eV) and cannot be populated by ISC from the S₁ (S₁→T₁) state or by internal conversion from the T₂ state (T₂→T₁). These results are consistent with the optical studies showing the absence of phosphorescence in TBP at 77 K.

### Geometry Optimization of TBP⊂Exbox⁴⁺ Using Different Basis Sets

The superstructure of TBP⊂Exbox⁴⁺ was optimized using three levels of theory: (i) B3LYP/3-21G (ii) B3LYP/6-31 G(d) (iii) APFD/6-31 G(d). The B3LYP functional does not provide a correct description of dispersion forces leading therefore to an overestimation of the distances (> 4 Å) between the TBP and the Exbipy²⁺ units of the ExBox⁴⁺. This large distance between the D and the A, will decrease the orbital overlap between the two moieties, offering a possibility to estimate the contribution of the locally excited (LE) states into transitions of similar energies. While in the case of APFD functional set, the dispersion forces are included, and the molecular optimized geometry is consistent with the crystal structures¹ of polyaromatic hydrocarbons incorporated into ExBox⁴⁺. In the APFD/6-31G(d) optimized molecular structure, the interplanar distance between the TBP and the Exbipy²⁺ unit are of 3.5 Å, corresponding to the Van Der Waal radii for [C···C] contact. A side-by-side comparison between these geometries illustrates how the extent of orbital overlap governs the formation of mixed states between the D and the A and the oscillator strength of the CT transitions.

### TD-DFT Calculation on TBP⊂Exbox⁴⁺ Using the B3LYP/LACV3P^{*+} Level of Theory

The superstructure of TBP⊂Exbox⁴⁺ was optimized at the B3LYP/3-21G level and time-dependent DFT (TD-DFT) calculations were carried out at the B3LYP/LACV3P^{*+} level on the optimized gas-phase geometry using Jaguar,⁵ considering a total of 130 excited states reaching into the upper end of the absorption spectrum (242.3 nm). Good agreement) was achieved between the calculated and experimental spectra. Ten low energy transitions were found with oscillator strengths > 0.001 which are tabulated in Table 10. Notably, there is one very weak transition at 435.64 nm with an oscillator strength that is 74 times less than the transition at 339.19 nm. The transitions at 384 and 374 nm which are relevant to the catalytic wavelength range (375-420 nm) are tabulated in Tables 11-15. All these transitions involve orbitals from both TBP and Exbox⁴⁺ components.

### TD-DFT Calculation on TBP⊂Exbox⁴⁺ Using the B3LYP/6-31G(d) Level of Theory

Time-dependent DFT calculations were carried out on the optimized gas-phase geometry of TBP⊂Exbox⁴⁺ at the B3LYP/6-31G(d) level of theory using the gaussian 16 package.⁴ These calculations were performed in order to investigate the singlet/triplet exciton transformation. The excited singlet (Sₙ) and triplet (Tₙ) states were investigated by time-dependent DFT (TD-DFT) on the optimized ground-state geometry using the same level of theory to investigate the vertical excitation energies. In order to gain insight into mixed transitions, natural transition orbitals (NTOs) were calculated to give a compact orbital representation for the electronic transformation within each state. Orbital overlap was calculated using the multi-wavefunction analysis software Multiwfn version 6.0⁶.

The singlet and triplet excited states of TBP⊂ExBox⁴⁺ consist of (Table 17) locally excited (LE) states residing either on the TBP or ExBox⁴⁺ components, charge transfer (CT) excited states and hybrid local charge-transfer (HLCT) excited states which are mixed states intermediate between a locally excited (LE) state and a charge-transfer (CT) state.⁷ The formation of mixed excited-states is consistent with the emission profile of the TBP⊂ExBox⁴⁺ complex which revealed the emergence of a lower energy band (520 nm) arising from exciton relaxation in the TBP⊂ExBox⁴⁺ complex (S₁, Table 16). The S₀→S₁ transition (Table 16) possesses a very weak oscillator strength and involves a pure CT transition from the TBP guest to the Exbox⁴⁺ host. Noteworthy, the lowest T₁ state (1.92 eV) is exclusively a LE state in TBP guest, while the S₀→T₂ (2.11 eV) and S₀-T₃ (2.13 eV) transitions are identical to the S₀→S₁ transitions having a pure CT transition from the host to the guest in the TBP⊂ExBox⁴⁺ complex. The extent of the HOMO to LUMO overlap is very small (7.9%) because of a larger interplanar distance (~ 4.2 Å) between the TBP and Exbipy²⁺ units (Van der Waal radii d_{C-C} = 3.5 Å). It was proposed⁸ previously that the minimum requirement for realizing exciton transformation is a matching of energy levels between two states, based on a thermal equilibrium between the singlet and triplet excited states. Although the exciton transformation channels S₁→T₂ and S₂→T₃ have a very small ΔE_{ST} (~ 0 eV), the weak molar absorption coefficient (small f) of the CT transitions in the TBP⊂ExBox⁴⁺ leads to a weak photosensitizing efficiency at λₑₓ = 450 nm. The lowest singlet excited state with non-negligible oscillator strength is the S₂ state (2.85 eV, ƒ= 0.0016) with pure CT character between HOMO (H) and LUMO+2 (L+2) (99%).

From Table 16, the S₀→T₆ transition configuration is very similar to that of S₀→S₂, both containing high HOMO→LUMO+2 components. The energy gap between the S₂ and T₆ states is very small (ΔE_{ST} = 0.0018 eV) and implies a facile S₂ →T₆ exciton transformation. The weak oscillator strength of the CT band, however, hampers efficient ¹O₂ generation at λₑₓ = 450 nm. The S₃ and S₄ excited states have non-negligible oscillator strengths (Table 16). Both the S₀→S₃ (3.20 eV, 387 nm, f = 0.07) and S₀→S₄ (3.25 eV, 380 nm, f = 0.03) are HLCT excited states involving both TBP→TBP, ExBox⁴⁺→ExBox⁴⁺ and TBP↔ExBox⁴⁺ transitions. Nevertheless, as a result of a large interplanar distance between TBP and the Exbipy²⁺ units, the extent of the orbital overlap is very small, and therefore the mixing of the orbitals in the host-guest complex is less significant compared to the APFT/6-31G(d) optimized molecular structure (FIG. 4).

The HONTOs and LUNTOs of all the hybridized singlet (S₃) and triplet states (T₇, T₈, T₉, T₁₂ and T₁₃) were investigated for the TBP⊂ExBox⁴⁺ complex. The singlet/triplet excited state pairs that have energy levels conducive to exciton transformation according to the energy gap law (|ΔE_{ST}| < 0.37 eV), have very similar HONTO and LUNTO distributions with the HONTO residing on the (TBP) donor moiety and the LUNTO residing on the (ExBox⁴⁺) acceptor moiety. A very small overlap between the HONTOs and LUNTOs were observed for these transitions. It is noteworthy that the low orbital overlaps between the D and A leads to LE and CT states while increasing orbital overlap between states of similar energies leads to HLCT states (FIGS. 5A-5C).

### Natural Transition Orbitals (NTOs) Computed at the B3LYP/6-31G(d) Level of Theory

Natural transition orbital (NTO) analysis was performed on the mixed excited states that involve components from both the TBP and ExBox⁴⁺ to elucidate the orbital migration in the singlet/triplet excited states.

### NTOs of the Triplet-States

The S₀→T₈ transition is a pure LE state involving the TBP guest exclusively. The S₀→T₉ transition is a pure CT state involving the TBP⊂ExBox⁴⁺ host-guest complex.

The S₀→T₁₂ transition is a pure CT state involving the TBP⊂ExBox⁴⁺ host-guest complex.

The S₀→T₁₃ transition is a LE state residing on the ExBox⁴⁺ host.

The S₀→T₁₄ and S₀→T₁₅ transitions are characteristic of LE behavior involving the ExBox⁴⁺ host exclusively, indicating the possibility of charge recombination in the p-xylene^{+•}-Exbipy^{3+•} complex, as it was already investigated experimentally.³

### TD-DFT Calculation on APFD/6-31G(d) Optimized TBP⊂Exbox⁴⁺ Structure Using B3LYP/6-31G(d) Level of Theory

The optimized molecular structure of the TBP⊂Exbox⁴⁺ using APFD/6-31G(d) basis set gave interplanar distances between the TBP host and Exbipy²⁺ guest of 3.5 Å. TD-DFT calculations were performed on this geometry using the B3LYP/6-31G(d) level of theory to investigate the singlet/triplet exciton transformation using the Gaussian 16 package.4 Good agreement was achieved between the positions of the calculated and experimental profiles. Natural transition orbital (NTO) analysis was performed to give a compact orbital representation for the electronic transformations to the excited states. The calculated UV-Vis absorption spectrum was plotted. The absorption band at 420 nm is associated to a CT transition between TBP and ExBox⁴⁺ while the band centered at 387 nm involves HLCT transitions for the S₂, S₃ and S₄ states.

The excited states S₀→S₂ and S₀→T₁ involves the same transition configuration (L→L+2 and L→L+3). The NTOs show that the T₁ is predominantly a LE transition while the S₂ is a HLCT transition. Although the orbital overlap between the HONTO and LUNTO for the S₂ and T₁ states are 38% and 90% respectively, the ΔE_{ST} (1.1 eV) is significantly larger than the limit of 0.37 eV for efficient intersystem crossing. Therefore, population of the T₁ state is more likely to arise from internal conversion mechanism from the upper Tₙ states. Particularly the ¹CT→³CT transformation (S₁→T₂ and S₁→T₃) display (FIGS. 5A-5C, Table 2) a very small ΔE_{ST} (0.0076 eV).

### DFT Calculations on the Ex^{2.2}Box⁴⁺ Using RB3LYP/6-31G^{*+}

DFT calculations on the geometry optimized structure of the Ex^{2.2}box⁴⁺ have been performed at the RB3LYP/6-31G^{*+} theory level using Jaguar.⁵ Both the HOMO and the LUMO are localized on the Ex^{2.2}bipy²⁺ units, therefore the photosensitizing properties originate from the locally excited triplet state of the Ex^{2.2}bipy²⁺ units.

### Photocatalysis Studies

Photocatalysts (0.002 mmol) 1 mol% were weighted in 17 mm × 83 mm glass microwave vials with a magnetic stir bar and sealed tightly by a crimper. Anhydrous MeOH or CD₃OD were utilized in order to monitor the catalysis kinetics using GC-FID and NMR spectroscopy, respectively. Solvent (1 mL) was injected through rubber cap and mixture was bubbled with O₂ gas for 20 min. Vials were left under 1 atm O₂ atmosphere. An internal standard, 1-bromo-3,5-difluorobenzene (10 µL, 0.08 mmol), and 2-chloroethyl ethyl sulfide (CEES) (23 µL, 0.2 mmol) were introduced through rubber cap by using a 50 micro liter syringe. Heterogeneous mixtures were sonicated for 10 sec before irradiation. Microwave vials were placed between two UV light-emitting diodes (LEDs) (max@395 nm, 500 mW.cm⁻²) over a magnetic stirrer and stirring was started at 700 rpm. Catalysis data points were collected using a 1 mL syringe at the beginning and after each irradiation time intervals. MeOH aliquots taken from the vials were transferred to a GC vial with dilution of CH₂Cl₂ (0.8 mL) and MeOH/ CD₃OD aliquots were diluted with CD₃OD (0.3 mL) in an NMR tube. Accordingly, ¹H and ¹³C NMR or GC-FID analysis of the samples were performed.

### Homogenous Catalysis (λₘₐₓ = 395 nm)

### Conversion Kinetic of CEES With ExBox•4Cl

ExBox•4Cl photocatalyst (1 mol%) was solubilized in CD₃OD in a microwave vial and sealed tightly with microwave vial cap with a septum by using a crimper. The reaction solution was bubbled with O₂ for 20 min. The reaction vial was left under O₂ atmosphere (P = 1 Atm) after O₂ purging. Internal standard (10 µL) and sulfur mustard simulant (23 µL) was added to the solution successively by using 50 µL syringe. Photo-irradiation at λₘₐₓ = 395 nm was achieved using two LEDs with power of 500 mW•cm⁻² while stirring with a small magnetic bar at 700 ppm. After 16 min photo-irradiation, the CEES is fully and selectively converted to the CEESO. The ¹³C NMR spectra confirms the formation of CEESO and the disappearance of the peaks of the CEES at 13.8, 25.4, 32.4 and 42.7 ppm and appearance of the peaks of the CEESO at 13.8, 25.4, 33.4, and 42.7.

### Conversion Kinetic of CEES With the Ex^{2.2}Box•4Cl

Very similar to preparation of ExBox•4Cl reaction mixture as described above, a mixture of IS, CEES and Ex^{2.2}Box•4Cl photocatalyst (1 mol%) were prepared in CD₃OD and the solution and the reaction was carried out under O₂ atmosphere (P = 1 Atm). Photoirradiation at λₘₐₓ = 395 nm was achieved using same LEDs and the photo-conversion of CEES was monitored by ¹H and ¹³C NMR spectroscopies. The overall conversion of the CEES was very slow and only reaches (FIG. 13) 46% after 60 min photoirradiation. In addition, the reaction was not selective, and only 23% of CEESO was formed whilst the other half of the conversion compounds were byproducts. MeOEES derivative was the second major product with 15% and its oxidized form (MeOEESO) was estimated to be 6%. Light-independent methanolysis of CEES to MeOEES was observed when the starting sample was left in MeOD solution for 7 h. Its relatively fast formation, however, in the presence of the Ex^{2.2}Box•4Cl is related to the decomposition of the Ex^{2.2}Box•4Cl cyclophanes upon photoirradiation. In fact, monitoring the decomposition of the Ex^{2.2}Box•4Cl using UV-vis (and ¹H NMR spectroscopies have shown dramatic changes of both absorption and ¹H NMR spectra. High resolution mass spectrometry confirms also the disappearance of the characteristic peaks of the M⁺ at 1203.2170 at photoirradiation at 395 nm.

### ¹H NMR Spectroscopy

On account of to the decomposition of Ex^{2.2}Box⁴⁺ with photo-irradiation, formation of MeOCEES increased significantly under UV light. Since photosensitization was also available during decomposition, some mono-oxidized version of MeOCEES (MeOCEESO) appeared during the reaction. In order to confirm the role of the photoirradiation in the increase of the reaction of the MeOH with CEES, we collected the ¹H NMR spectrum of CEES left in a solution of Ex^{2.2}Box⁴⁺ in MeOH for 7 h (See spectrum labelled 7 h (no irradiation). CEES is relatively stable in methanol and confirms the role of the instability of the Ex^{2.2}Box⁴⁺ in the formation of MeOEES and MeOEESO.

### Conversion Kinetic of CEES With TBP⊂ExBox•4Cl

Photocatalytic oxidation of CEES using TBP⊂ExBox•4Cl was realized in the same fashion as with the former homogeneous photocatalysts. After 9 min photoirradiation, the CEES is fully and selectively converted to the CEESO. Seemingly, the rate of conversion of the CEES with the supramolecular photocatalyst TBP⊂ExBox•4Cl is 50% faster than ExBox•4Cl, indicative of the efficiency of the singlet to triplet transformation in such supramolecular complexes. ¹H NMR and ¹³C NMR confirmed the oxidation of CEES to CEESO.

### Heterogenous Catalysis

ExBox·PSS, Ex^{2.2}Box•PSS and TBP⊂ExBox•PSS photocatalyst composites (1 mol%) were suspended in a solution of MeOH in a similar way to that explained above for the homogenous catalysts. The solution was saturated with O₂ and the reaction was carried out under O₂ atmosphere (P = 1 bar). Dispersions were sonicated for 10 sec and then photoirradiation at λₘₐₓ = 395 nm was conducted using LEDs while stirring at 700 rpm. All samples were filtrated with 10 µm pore sized PFFE syringe filter using CH₂Cl₂ (0.8 ml) and filtrate was collected in GC vials and GC-FID analysis of the samples were conducted. Additional control experiments with Na·PSS, TBP and TBP•PSS have been carried out using similar reaction conditions to unravel the contribution of the PSS anionic matrix as well the TBP to the photocatalytic performances. FIG. 14 shows that the elimination of CEES is negligible with Na·PSS and MeOEES is formed at 10% after 60 min photoirradiation. These results are consistent with the spectroscopic investigations showing that the Na·PSS is optically inactive above 260 nm. The TBP and TBP-Na•PSS composite exhibit similar photocatalytic performance with CEES conversion of ~50% after 60 min photo-irradiation. Photosensitizing efficiency of the TBP is weak on account of the insolubility and aggregation of the compound despite of the presence of Br atoms which may enhance the intersystem crossing behavior.⁹ Therefore, the high efficiency of the TBP⊂ExBox•PSS photosensitizers implies the existence of other pathways of singlet to triplet transportation that lead to the increase of the photocatalytic efficiency.

### Conversion Kinetic of the CEES Using ExBox·PSS Composite

The photocatalysis of CEES with ExBox·PSS composite was carried out for 60 min under 395 nm photoirradiation. The conversion of the CEES was observed to be only 50% at 60 min indicating the weak photosensitizing character of the ExBox⁴⁺ in the solid state as the result of the stabilization of the singlet state evident with the increase in the singlet lifetime. Both ¹H and ¹³C NMR spectra show the presence of both CEES and CEESO. Despite of low conversions, the reaction was highly selective towards the sulfoxide product. No harmful sulfone product was observed.

### Conversion Kinetic of CEES With the Ex^{2.2}Box•PSS Composite

The photocatalysis of CEES with the Ex^{2.2}Box•PSS composite was carried out (FIG. 14) for 70 min under 395 nm photoirradiation. The conversion kinetic of the CEES to CEESO was monitored by GC-FID. The conversion of the CEES was 100% completed. ¹³C NMR spectra shows that the formation of CEESO is 100% selective. These results are in contrast to the homogenous catalysis which have shown the decomposition of the Ex^{2,2}Box⁴⁺ cyclophane under the photocatalytic conditions. Clearly the PSS matrices provide additional stability to the cyclophanes, leading to both the increase in the catalytic kinetics, better selectivity and high stability of the photosensitizer.

### Conversion Kinetics of CEES With TBP⊂ExBox•PSS Composite

The conversion kinetics of the CEES to CEESO was monitored by GC-FID. The photocatalysis of CEES with the TBP⊂ExBox•PSS composite was carried out for 60 min under 395 nm photoirradiation. The conversion of the CEES was 100% completed after 18 min photoirradiation. ¹³C NMR spectra show that the formation of CEESO is 100% selective. It is noteworthy that over irradiation up to 60 min does not lead to the formation of the sulfone derivative (CEESO₂) confirming the selective nature of the TBP⊂ExBox•PSS composite.

### Photocatalysis of the CEES With TBP⊂ExBox•PSS Using White Light Photoirradiation (λₘₐₓ =450 nm)

The kinetic of the conversion of the CEES to CEESO was monitored by GC-FID. The photocatalysis of CEES with the TBP⊂ExBox•PSS composite was carried out for 35 min under 450 nm photoirradiation. The conversion of the CEES is very slow (18%) (FIG. 15) indicative weak contribution of the low lying CT states in the TBP⊂ExBox⁴⁺ host-guest complex in the enhancement of the catalytic properties. These results confirm the role of the HLCT states for efficient S-T transformation, populating the low-lying the T₁ state and enhancing therefore, the photosensitizing properties. ¹³C NMR spectrum show that the formation of CEESO is the major product.

### Conversion Kinetics of CEES With TBP

After preparation catalysis reaction in MeOH (1 mL) using 1 mol% of the TBP, which is not soluble in MeOH, as described above, photoirradiation of TBP resulted in two major products, CEESO and MeOEES in 60 min. Among the products, the sulfoxide version of MeOEES (MEOEESO) was also observed. ¹³C NMR spectra of the products at 60 min confirms the GC-FID results. Overall selectivity (FIG. 63) of CEESO was calculated to be 58%. No harmful sulfone derivatives were observed.

### Conversion Kinetics of CEES With TBP•PSS

Photocatalytic reaction of the TBP•PSS (1 mol%) was performed (FIG. 14) in MeOH as described above and 60% conversion was observed in 60 min. Interestingly, Na·PSS increased the selectivity of the catalyst for CEESO up to 97% and no harmful sulfone derivatives were observed. Negligible amounts of MeOEES are formed on account of the self-methanolysis of CEES in MeOH in 60 min.

### Conversion Kinetics of CEES With Na·PSS

Na·PSS was used as a photocatalyst to oxidize CEES. According to ¹³C NMR spectra of reaction solution at 60 min, no CEESO formation was observed. According to GC-FID and NMR analysis, however, 10% CEES was converted (FIG. 14) into MeOEES due to self-methanolysis in MeOH solution. Conversion mount of self methanolysis of CEES in MeOH depends on how long CEES stays in the MeOH solution. Since CD₃OD was used as NMR solvent, the time of the solution passing in NMR autosampler queue may increase the formation of MeOEES. It is noteworthy to mention here that once CEES is oxidized to CEESO, CEESO no longer react with MeOH to form MeOEESO.

### Leach Test TBP⊂ExBox•PSS

The leach test was conducted (FIG. 16) following a similar procedure utilized to test the photocatalysis performances of the TBP⊂ExBox•PSS composite. The measurement was repeated by the second sample collection point at which photo-irradiation was ceased at 50% conversion (5 min photoirradiation) and the reaction mixture was filtered with a syringe filter with 0.13 µm-pore size. Filtrate was transferred to another 17 mm × 83 mm glass microwave vials and the vial promptly sealed by a crimper. The sealed vial was purged with oxygen through syringe needles for 15 min and left under 1 bar oxygen at the end of the purge. The photo-irradiation was continued, and small amounts of aliquot were taken from the reaction mixture at the desired time intervals. The aliquots were diluted with CH₂Cl₂ (0.8 mL) before they were introduced to GC-FID.

### TABLES

TABLE 1

Fluorescence parameters of the ExBox·PSS, TBP, and TBP⊂ExBox•PSS at 298 K.

| Sample | λₑₘ (nm) | τ₁ (ns) [Amplitude%] | τ₂ (ns) [Amplitude%] | τ₃ (ns) [Amplitude %] |
|---|---|---|---|---|
| TBP⊂ExBox•PSS | 440 515 | 0.137 [37.97] 0.3512 [13.39] | 1.32 [27.7] 2.027 [56.8] | 4.031 [34.33] 6.14 [29.81] |
| ExBox•PSS | 480 | 0.22[20.27] | 1.40 [42.89] | 8.23 [36.84] |
| TBP^{a} | 439 | 0.11 [40.09] | 0.60[8.55] | 10.27 [51.36] |

^{a} Solubilized in MePh

TABLE 2

Excitation energy (E in eV), oscillator strength (f), transition configuration of S0 → Sn and S0 → Tn for exciton transformation,a and energy gap of the singlet-triplet splitting (DEST).b These calculation are based of the APFD-6-31G(d) geometry optimized molecular structure

| Sₙ | E (eV) | Oscillator Strength (f) | Transition Configuration (%) | Tₙ | E (eV) | Transition Configuration (%) | ΔE_{ST} | ΔE_{ST} (eV) |
|---|---|---|---|---|---|---|---|---|
| 1 | 2.24 | 0.0004 | H→L+1(99.7%) | 1 | 1.89 | H→L+2 (88%), H→L+3 (5.2%) | ^{2,1}ΔE_{ST} | 1.0971 |
| 2 | 2.99 | 0.0097 | H→L+2(33.8%), H→L+3(62.6%) | 2 | 2.19 | H→L(99.6%) | ^{1,2}ΔE_{ST} | 0.049 |
| 3 | 3.11 | 0.0131 | H-1→L+1(14.1%) | 3 | 2.24 | H→L+1 (98.4%) | ^{1,3}ΔE_{ST} | 0.0076 |
| | | | H→L+2 (45.3%), H→L+3 (31.1%) | 4 | 2.56 | H-4→L+1(29.8%),H-1→L(47.5%) | ^{3,4}ΔE_{ST} | 0.5546 |
| 4 | 3.20 | 0.2478 | H-1→L+1(80.5%), H→L+2 (8.8%) | 5 | 2.57 | H-4→L+1(32.8%), H-1→L(43.9%) | ^{3,5}ΔE_{ST} | 0.5462 |
| 5 | 3.41 | 0.0017 | H-3→L(94.5%) | 6 | 2.96 | H→L+3(80.2%) | ^{3,6}ΔE_{ST} | 0.1573 |
| 6 | 3.46 | 0.0012 | H-1→L+2(28.7%) | 7 | 3.10 | H→L+4(97.3%) | | |
| | | | H→L+5(37.6%), H→L+7(13.5 %) | 8 | 3.19 | H-9→L+1 (19.2%) | ^{4,8}ΔE_{ST} | 0.0065 |
| 7 | 3.51 | 0.0346 | H-5→L(87.0%), H-4→L (6.0%) | | | H-7→L(29.1%), H-1→L+1(29.1%) | | |
| 8 | 3.55 | 0.0052 | H-6→L+1 (73.2%), H-4→L(17.3%) | 9 | 3.19 | H-9→L(22.5%) | ^{4,9}ΔE_{ST} | 0.0064 |
| 9 | 3.57 | 0.2771 | H-6→L+1(18.5%) | | | H-7--7L+ 1(20.7%), H-I--7L(12.4%) | | |
| 10 | 3.65 | 0.323 | H-7→L (30.2%), H-4→L (34.3%) H-4→L(28.7%), H-7→L (53.7%), H-9→L+1 (5.9%) | 10 | 3.21 | H-1→L+2(10.6%), H→L+3(10.4%) H→L+5(38.8%), H→L+7(17.8%) | ^{3,10}ΔE_{ST} | 0.098 |

^{a}H and L represents respectively HOMOs and LUMOs. ^{b} The most similar and energy close ΔE_{ST} are highlighted with the same color.

TABLE 3

Homogenous photocatalysis parameters using 1 mol % of photocatalyst at 395 nm photoirradiation in CD3OD-d4

| Catalyst | Irradiation time (min) | Total conversion | Full conversion | t_{½} (min) | (CEESO) Selectivity |
|---|---|---|---|---|---|
| ExBox•4Cl | 20 | 100 | 16 | 7 | 97 |
| Ex^{2,2}Box•4Cl | 120 | 60 | / | 79 | 34 |
| TBP⊂ExBox•4Cl | 27 | 100 | 9 | 3.5 | 97 |

TABLE 4

Heterogenous photocatalysis parameters using 1 mol % of photocatalyst obtained at 395 nm photoexcitation in MeOH

| Catalyst | Total Photoirradiation | Total conversion | Time to full conversion | Half-life t_{½} (min) | % Selectivity (CEESO) |
|---|---|---|---|---|---|
| ExBox•PSS | 60 | 50 | / | 60 | >99 |
| Ex^{2,2}Box•PSS | 60 | 100 | 54 | 18 | >99 |
| TBP⊂ExBox•PSS | 25 | 100 | 20 | 5 | >99 |
| TBP•PSS | 60 | 60 | / | 52 | 97 |
| TBP | 60 | 50 | / | 60 | 58 |
| Na•PSS | 60 | 10 | / | / | 0 |

TABLE 5

Emission quantum yields (Φ_{F}) at 298 K

| Sample | λₑₓ / nm | Φ_{F} / % | Excitation Range / nm | Emission Range / nm |
|---|---|---|---|---|
| TBP⊂ExBox•PSS | 380 | 2.18 | 360-399 | 400-700 |
| TBP⊂ExBox•PF₆ | 378 | 3.15 | 358-399 | 400-700 |
| ExBox•PSS | 380 | 1.32 | 360-399 | 400-700 |
| TBP | 380 | 1.62 | 360-399 | 400-700 |

TABLE 6

Calculated Stokes shifts in the solid-state at 298 K

| Sample | λₑₓ | λₑₘ | Stokes shifts ΔE / eV |
|---|---|---|---|
| TBP⊂ExBox•PSS | 362 | 522 | 1.07 |
| ExBox•PSS | 390 | 469 | 0.54 |
| TBP | 374 | 401 | 0.22 |

TABLE 7

Computed orbital energies (eV) of the TBP⊂ExBox⁴⁺ complex (APFD/B3LYP/6-31G(d))

| | HOMO | HOMO-1 | HOMO-2 | LUMO | LUMO+1 | LUMO+2 | LUMO+3 |
|---|---|---|---|---|---|---|---|
| TBP⊂ExBox⁴⁺ | -6.45 | -7.33 | -7.57 | -3.71 | -3.66 | -3.09 | -2.90 |

TABLE 8

B3LYP/6-31G(d) computed transitions for TBP with oscillator strengths (ƒ) greater than 0.05

| Transition | Wavelength(nm) | ƒ | Transition dipole moment (Debye) |
|---|---|---|---|
| 1 | 373.89 | 0.4620 | 5.6862 |
| 4 | 282.78 | 0.3566 | 3.3203 |
| 13 | 239.26 | 0.5709 | 4.4968 |

TABLE 9

Contributions to the 373.89 nm excitation (S₀→S₁ transition)

| Excitation | | X coeff. |
|---|---|---|
| HOMO-1 | => LUMO+1 | -0.15045 |
| HOMO | => LUMO | 0.68945 |

TABLE 10

B3LYP/LACV3P*+ computed transitions for the TBP⊂Exbox⁴⁺ with oscillator strengths (ƒ) greater than 0.001

| Transition | Wavelength(nm) | ƒ | Transition dipole moment (Debye) |
|---|---|---|---|
| 1 | 550.97 | 0.0011 | 0.35 |
| 2 | 435.64 | 0.0075 | 0.83 |
| 3 | 384.33 | 0.0703 | 2.40 |
| 4 | 374.64 | 0.0581 | 2.15 |
| 5 | 356.30 | 0.0066 | 0.70 |
| 6 | 352.85 | 0.0071 | 0.73 |
| 7 | 339.65 | 0.1817 | 3.62 |
| 8 | 339.19 | 0.5566 | 6.33 |
| 9 | 337.65 | 0.0636 | 2.14 |
| 10 | 335.99 | 0.1508 | 3.28 |

TABLE 11

DFT computed orbital energies (eV) of the TBP⊂ExBox⁴⁺ optimized geometries based on B3LYP/LACV3P*⁺ calculations

| | HOMO | HOMO-1 | HOMO-2 | LUMO | LUMO+1 | LUMO+2 | LUMO+3 |
|---|---|---|---|---|---|---|---|
| TBP⊂ExBox⁴⁺ | -6.32 | -7.39 | -7.50 | -3.59 | -3.00 | -2.92 | -2.81 |

TABLE 12

Contributions to the 550.97 nm excitation

| Excitation | | X coeff. |
|---|---|---|
| HOMO | => LUMO+1 | 0.99919 |

TABLE 13

Contributions to the 435.64 nm excitation

| Excitation | | X coeff. |
|---|---|---|
| HOMO | => LUMO+2 | 0.97498 |
| HOMO | => LUMO+4 | 0.19656 |

TABLE 14

Contributions to the 384.33 nm excitation

| Excitation | | X coeff. |
|---|---|---|
| HOMO-5 | => LUMO | -0.18822 |
| HOMO-5 | => LUMO+1 | -0.18334 |
| HOMO-1 | => LUMO+9 | -0.11237 |
| HOMO | => LUMO+2 | -0.16781 |
| HOMO | => LUMO+4 | 0.91896 |

TABLE 15

Contributions to the 374.33 nm excitation

| Excitation | | X coeff. |
|---|---|---|
| HOMO-4 | => LUMO | 0.14274 |
| HOMO-1 | => LUMO | -0.98102 |

TABLE 16

Excitation energy (E in eV), oscillator strength (ƒ), transition configuration of S₀ → Sₙ and S₀ → Tₙ for exciton transformation, and energy gap of the singlet-triplet splitting (ΔE_{ST}) calculated from optimized gas-phase geometry of TBP⊂Exbox⁴⁺ at the B3LYP/6-31G(d) level of theory

| Sₙ | E (eV) | Oscillator strength (ƒ) | Transition configuration (%) | Tₙ | E (eV) | Transition configuration (%) | ΔE_{ST} | ΔE_{ST} (eV) |
|---|---|---|---|---|---|---|---|---|
| 1 | 2.14 | 0.0005 | H→L+1(99.9%) | 1 | 1.92 | H→L+4 (94%), L+4→H (2.5%) | ^{3,1}ΔE_{ST} | 1.28 |
| 2 | 2.85 | 0.0016 | H→L+2(99.0%) | 2 | 2.11 | H→L(99.8%) | ^{1,2}ΔE_{ST} | 0.03 |
| 3 | 3.20 | 0.0691 | H-5→L(4.98%), H-4→L+1 (5.26%), H→L+4(85.49%) | 3 | 2.13 | H→L+1(99.9%) | ^{1,3}ΔE_{ST} | 0.01 |
| | | | | 4 | 2.65 | H-8→L(8.5%), H-7→L+1(2.1%) H-5→L+1(37.9%), H- 4→L(24.9%) H-1→L(13.4%) | ^{4,4}ΔE_{ST} | 0.61 |
| 4 | 3.26 | 0.0302 | H-1L+1(97.41%) | | | | | |
| | | | | 5 | 2.66 | H-8→L+1(8.5%), H-7→L(2.2%), H-5→L(39.4%), H-4→L+1(24%) H-1→L+1(12.7%) | ^{4,5}ΔE_{ST} | 0.6 |
| 5 | 3.42 | 0.0006 | H-3→L(95.9%), H-2→L+2 (3.9%) | | | | | |
| 6 | 3.5 | 0.0064 | H-1→L+5(23.4%), H→L+7 (44.2%), H→L+9(29.5%) | 6 | 2.85 | H→L+2(98.9%) | ^{2,6}ΔE_{ST} | 0 |
| 7 | 3.62 | 0.0052 | H-10→L+1(5.4%), H- 7→L(90.5%) | 7 | 2.9 | H→L+3(98.9%) | ^{5,7}ΔE_{ST} | |
| | | | | 8 | 3.14 | H-1→L+4(3.2%), H→L+7(37.7%) H→L+9(57.2%) | ^{5,8}ΔE_{ST} | |
| 8 | 3.62 | 0.269 | H-9→L+1(8.16%), H-6→L+3 (2.26%), H-4→L+1(73.79%) H→L+5(11.68%) | | | | | |
| | | | | 9 | 3.24 | H-5→L+1(3.6%), H-4→L(9.6%) H-1→L(83.1%) | ^{4,9}ΔE_{sT} | 0.02 |
| 9 | 3.63 | 0.0191 | H-6→L+1(8.59%), H→L+5 (85.26%) | | | | | |
| | | | | 12 | 3.26 | H-5→L(3.5%), H-4→L+1 (10.0%) H-1→L+1(83.1%) | ^{4,12}ΔE_{S} T | 0 |
| 10 | 3.65 | 0.0377 | H-9→L+2(2.73%). H-6→L (91.48%) | | | | | |

TABLE 17

DFT computed orbital energies (eV) of the TBP ⊂ ExBox⁴⁺ optimized geometries based on B3LYP/6-31Gdp calculations

| | HOMO | HOMO-1 | HOMO-2 | LUMO | LUMO+1 | LUMO+2 | LUMO+3 |
|---|---|---|---|---|---|---|---|
| TBP | -5.80 | -6.93 | -7.36 | -2.33 | -1.51 | -0.71 | -0.65 |
| TBP⊂ExBox⁴⁺ | -6.09 | -7.21 | -7.33 | -3.48 | -3.45 | -2.75 | -2.69 |

### REFERENCES

S. B. Brown, E. A. Brown, I. Walker, Lancet Oncol. 2004, 5, 497.

R. C. Evans, P. Douglas, C. J. Winscom, Coord. Chem. Rev. 2006, 250, 2093.

D. M. Arias-Rotondoa, J. K. McCusker, Chem. Soc. Rev. 2016, 45, 5803.

X.-D. Wang, O. S. Wolfbeis, Chem. Soc. Rev. 2014, 43, 3666.

[⁵] J. Zhou, Q. Liu, W. Feng, Y. Sun, F. Li, Chem. Rev. 2015, 115, 395.

a) M. A. El-Sayed, J. Chem. Phys. 1974, 60, 4502. (b) C. M. Marian, Wiley Interdiscip. Rev. Comput. Mol. Sci. 2012, 2, 187.

M. A. Filatov, S. Karuthedath, P. M. Polestshuk, H. Savoie, K. J. Flanagan, C. Sy, E. Sitte, M. Telitchko, F. Laquai, R. W. Boyle, M. O. Senge, J. Am. Chem. Soc. 2017, 139, 6282.

M. A. Filatov, S. Karuthedath, P. M. Polestshuk, S. Callaghan, K. J. Flanagan, T. Wiesner, F. Laquai, M. O. Senge, ChemPhotoChem. 2018, 2, 606.

R. Chen, Y. Tang, Y. Wan, T. Chen, C. Zheng, Y. Qi, Y. Cheng, W. Huang, Sci. Rep. 2017, 7, 6225.

a) Y. Liu, A. J. Howarth, J. T. Hupp, O. K. Farha, Angew. Chem. Int. Ed. Engl. 2015, 54, 9001; b) F. Jensen, A. Greer, E. L. Clennan, J. Am. Chem. Soc. 1998, 18, 4439.

a) A. Atilgan, T. Islamoglu, A. J. Howarth, J. T. Hupp, O. K. Farha, ACS Appl. Mater. Interfaces 2017, 9, 2455; (b) H. Wang, G. W. Wagner, A. X. Lu, D. L. Nguyen, J. H. Buchanan, P. M. McNutt, C. J. Karwacki, ACS Appl. Mater. Interfaces 2018, 10, 18771; c) A. J. Howarth, C. T. Buru, Y. Liu, A. M. Ploskonka, K. J. Hartlieb, M. McEntee, J. J. Mahle, J. H. Buchanan, E. M. Durke, S. S. Al-Juaid, J. F. Stoddart, J. B. DeCoste, J. T. Hupp, O. K. Farha, Chem. Eur. J. 2017, 23, 214; d) C. F. Pereira, Y. Liu, A. Howarth, F. Figueira, J. Rocha, J. T. Hupp, O. K. Farha, J. P. C. Tomé, F. A. A. Paz, ACS Applied Nano Materials 2019, 2, 465; e) Y. Liu, C. T. Buru, A. J. Howarth, J. J. Mahle, J. H. Buchanan, J. B. DeCoste, J. T. Hupp, O. K. Farha, J. Mater. Chem. A 2016, 4, 13809.

H. Wang, G. W. Wagner, A. X. Lu, D. L. Nguyen, J. H. Buchanan, P. M. McNutt, C. J. Karwacki, ACS Appl. Mater. Interfaces 2018, 10, 18771.

R. M. Young, S. M. Dyar, J. C. Barnes, M. Juríček, J. F. Stoddart, D. T. Co, M. R. Wasielewski, J. Phys. Chem. A 2013, 117, 12438.

S. M. Dyar, J. C. Barnes, M. Juríček, J. F. Stoddart, D. T. Co, R. M. Young, M. R. Wasielewski, Angew. Chem. Int. Ed. 2014, 53, 5371.

R. M. Young, S. C. Jensen, K. Edme, Y. Wu, M. D. Krzyaniak, N. A. Vermeulen, E. J. Dale, J. F. Stoddart, E. A. Weiss, M. R. Wasielewski, D. T. Co, J. Am. Chem. Soc. 2016, 138, 6163.

S. T. J. Ryan, J. Del Barrio, I. Ghosh, F. Biedermann, A. I. Lazar, Y. Lan, R. J. Coulston, W. M. Nau, O. A. Scherman, J. Am. Chem. Soc. 2014, 136, 9053.

S. T. J. Ryan, R. M. Young, J. J. Henkelis, N. Hafezi, N. A. Vermeulen, A. Hennig, E. J. Dale, Y. Wu, M. D. Krzyaniak, A. Fox, W. M. Nau, M. R. Wasielewski, J. F. Stoddart, O. A. Scherman, J. Am. Chem. Soc. 2015, 137, 15299.

D. L. Dexter, J. Chem. Phys. 1953, 21, 836.

a) Y. Hou, X. Zhang, K. Chen, D. Liu, Z. Wang, Q. Liu, J. Zhao, A. Barbon, J. Mater. Chem. C, 2019, 7, 12048; b) Z. Wang, M. Ivanov, Y. Gao, L. Bussotti, P. Foggi, H. Zhang, N. Russo, B. Dick, J. Zhao, M. Di Donato, G. Mazzone, L. Luo, M. Fedin, Chem.Eur.J. 2020, 26, 1091.

D. Kovalev, M. Fujii, Adv. Mater. 2005, 17, 2531.

P. R. Symmers, M. J. Burke, D. P. August, P. I. T. Thomson, G. S. Nichol, M. R. Warren, C. J. Campbell, P. J. Lusby Chem. Sci. 2015, 6, 756.

J. C. Barnes, M. Juríček, N. L. Strutt, M. Frasconi, S. Sampath, M. A. Giesener, P. L. McGrier, C. J. Bruns, C. L. Stern, A. A. Sarjeant, J. F. Stoddart, J. Am. Chem. Soc. 2013, 135, 183.

K. C. Kim, T.-U. Yoon, Y.-S. Bae, Microporous Mesoporous Mater. 2016, 224, 294.

M. M. Khan, G. Bengtson, S. Neumann, M. Rahman, V. Abetz, V. Filiz, RSC Adv. 2014, 4, 32148.

a) M. M. Cetin, Y. Beldjoudi, I. Roy, O. Anamimoghadam, Y.-J Bae, R. M. Young, M. D. Krzyaniak, C. L. Stern, D. Philp, F. M. Alsubaie, M. R. Wasielewski, J. F. Stoddart, J. Am. Chem. Soc. 2019, 141, 18727; b) K. Cai, M. C. Lipke, Z. Liu, J. Nelson, T. Cheng, Y. Shi, C. Cheng, D. Shen, J.-M. Han, S. Vemuri, Y. Feng, C. L. Stern, W. A. Goddard, M. R. Wasielewski, J. F. Stoddart, Nat. Commun. 2018, 9, 5275.

a) Y. Luan, N. Zheng, Y. Qi, J. Tang, G. Wang, Catal. Sci. Technol. 2014, 4, 925; b) W. Zhang, B. Zheng, W. Shi, X. Chen, Z. Xu, S. Li, Y. R. Chi, Y. Yang, J. Lu, W. Huang, F. Huo, adv. Mater. 2018, 30, 1800643; c) J. Duan, S. Chen, C. Zhao, Nat. Communi. 2017, 8, 15341; d) S. Zhao, Y. Wang, J. Dong, C.-T. He, H. Yin, P. An, K. Zhao, X. Zhang, C. Gao, L. Zhang, J. Lv, J. Wang, J. Zhang, A. M. Khattak, N. A. Khan, Z. Wei, J. Zhang, S. Liu, H. Zhao, Z. Tang, Nat. Energy 2016, 1, 16184.

a) Z. E. X. Dance, S. M. Mickley, T. M. Wilson, A. B. Ricks, A. M. Scott, M. A. Ratner, M. R. Wasielewski, J. Phys. Chem. A 2008, 112, 4194; b) C. V. Suneesh, K. R. Gopidas, J. Phys. Chem. C 2010, 114, 18725; c) Y. Dong, A. A. Sukhanov, J. Zhao, A. Elmali, X. Li, B. Dick, A. Karatay, V. K. Voronkova, J. Phys. Chem. C 2019, 123, 22793.

Y. Wu, J. Zhou, B. T. Phelan, C. M. Mauck, J. F. Stoddart, R. M. Young, M. R. Wasielewski, J. Am. Chem. Soc. 2017, 139, 14265.

The total decay rate (kₜₒₜ) is the sum of the radiative (k_{rad}) and nonradiative (k_{nrad}) rates: kₜₒₜ = k_{rad} + k_{nred}. The emission quantum yield is given by this equation: Φ_{F} = k_{rad}/kₜₒₜ

R. A. Marcus, J. Chem. Phys. 1956, 24, 966; b) P. Vath, M. B. Zimmt, J. Phys. Chem. A 2000, 104, 2626; c) J. T. Hupp, Y. Dong, R. L. Blackbourn, H. Lu, J. Phys. Chem. 1993, 97, 3278.

A. Austin, G. A. Petersson, M. J. Frisch, F. J. Dobek, G. Scalmani, K. Throssell, J. Chem. Theory Comput. 2012, 8, 4989.

M. J Frisch, G. W. Trucks, H. B. Schlegel, G. E. Scuseria, M.A. Robb, J. R. Cheeseman, G. Scalmani, V. Barone, G. A. Petersson, H. Nakatsuji, X. Li, M. Caricato, A. V. Marenich, J. Bloino, B. G. Janesko, R. Gomperts, B. Mennucci, H. P. Hratchian, J. V. Ortiz, A. F. Izmaylov, J. L. Sonnenberg, Williams, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. A. Montgomery Jr., J. E. Peralta, F. Ogliaro, M. J. Bearpark, J. J. Heyd, E. N. Brothers, K. N. Kudin, V. N. Staroverov, T. A. Keith, R. Kobayashi, J. Normand, K. Raghavachari, A. P. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman, D. J. Fox, Gaussian 16 Rev. C.01, Wallingford, CT, 2016.

Y. Y. Pan, J. Huang, Z. M. Wang, S. T. Zhang, D. W. Yu, B. Yang, Y. G. Ma, RSC Advances 2016, 6, 108404.

K. Hasharoni, H. Levanon, S. R. Greenfield, D. J. Gosztola, W. A. Svec, M. R. Wasielewski, J. Am. Chem. Soc. 1996, 118, 10228.

R. L. Martin, J. Chem. Phys. 2003, 118, 4775.

a) J. J. Liang, C. L. Gu, M. L. Kacher, C. S. Foote, J. Am. Chem. Soc. 1983, 105, 4717; b) R. Limvorapitux, H. Chen, M. L. Mendonca, M. Liu, R. Q. Snurr, S. T. Nguyen, Catal. Sci. Technol. 2019, 9, 327.

Y. Beldjoudi, I. Osorio-Roman, M. A. Nascimento, J. M. Rawson, J. Mater. Chem. C 2017, 5,2794.

1. A photocatalyst comprising a nanocomposite, the nanocomposite comprising an admixture of a polymeric matrix and a macrocycle, and a polyaromatic guest, wherein the macrocycle and the polyaromatic guest form a host-guest complex.

2. The photocatalyst of claim 1, wherein the polyaromatic guest and the macrocycle form a donor-acceptor dyad.

3. The photocatalyst of claim 1, wherein the macrocycle is a cationic cyclophane.

4. The photocatalyst of claim 3, wherein the cyclophane is ExBox⁴⁺ or Ex^{2.2}Box⁴⁺.

5. The photocatalyst of claim 1, wherein the polymeric matrix comprises an anionic polymer.

6. The photocatalyst of claim 5, wherein the polymeric matrix comprises polystyrene sulfonate (PSS).

7. The photocatalyst of claim 1, wherein the polyaromatic guest is a substituted pyrene.

8. The photocatalyst of claim 7, wherein the substituted pyrene is1,3,5,8-tetrabropyrene (TBP).

9. The photocatalyst of claim 1, wherein the macrocycle is a cationic cyclophane, the polymeric matrix comprises an anionic polymer, and the polyaromatic guest is a substituted pyrene.

10. The photocatalyst of claim 9, wherein the cyclophane is ExBox⁴⁺ or Ex^{2.2}Box⁴⁺, the polymeric matrix comprises polystyrene sulfonate (PSS), and the substituted pyrene is 1,3,5,8-tetrabropyrene (TBP).

11. The photocatalyst of claim 1 wherein the polyaromatic guest and the macrocycle absorb similar radiation wavelengths.

12. The photocatalyst of claim 1, wherein he polyaromatic guest and the macrocycle are configured for spin-orbit charge-transfer intersystem crossing.

13. The photocatalyst of claim 1, wherein the photocatalyst has a similar CT and T₁ state.

14. The photocatalyst of claim 1, wherein the polyaromatic guest and/or the macrocycle has a low lying triplet state.

15. The nanocomposite according to claim 1.

16. A fiber comprising the photocatalyst or nanocomposite according to claim 1.

17. A fabric comprising the photocatalyst or nanocomposite according to claim 1.

18. A nanoparticle comprising the photocatalyst or nanocomposite according to claim 1.

19. A method for photocatalytic oxidation of a reactive substrate, the method comprising contacting the photocatalyst or the nanocomposite according to claim 1 with a reactive substrate and irradiating the photocatalyst or the nanocomposite in the presence of the reactive substrate, thereby oxidizing the reactive substrate.

20. (canceled)

21. (canceled)

22. (canceled)

23. A method for the generation of singlet oxygen (¹O₂), the method comprising irradiating the photocatalyst or the nanocomposite according to claim 1 in the presence of an oxygen source.

24. (canceled)

25. (canceled)

26. (canceled)

27. A method for sequestering an environmental contaminant, the method comprising contacting the photocatalyst or the nanocomposite according to claim 1 with the environmental contaminant under conditions suitable to the adsorption of the environmental contaminant.

28. (canceled)

29. (canceled)

30. A method for preparing a nanocomposite or a photocatalyst, the method comprising:
providing a first macrocycle solution comprising a macrocycle, a macrocycle solvent, and a first counterion,
preparing a second macrocycle solution comprising the macrocycle, the solvent, and a second counterion, wherein the second counterion is different than the first counterion, providing a polymer solution comprising a polymer and a polymer solvent, mixing the second macrocycle solution and the polymer solution, thereby precipitating the nanocomposite or the photocatalyst from solution.

31. (canceled)

32. (canceled)

33. (canceled)
